SEQUENCE LISTING (74 sequences)

SEQ ID NO: 1 - DNA, 351 bp, Artificial Sequence
  Description of Artificial Sequence Synthetic polynucleotide
caggtgcagctgaaggagtcaggacctggcctggtggcgccctcacagagcctgtccatcacttgcactgtctctgggttttcattaatcagctatggtgtagactgggttcgccagcctccaggaaagggtctggagtggctgggagtaatatggggtggtggaaatacaaattataattcatctctcatgtccagactgagcatcagcaaagacaactccaagagccaagttttcttaaaaatgaacagtctgcaaactgatgacacagccatgtactactgtgccaaaactgggaccggatatgctttggagtactggggtcaaggaacctcagtcaccgtctcctcc

SEQ ID NO: 2 - DNA, 321 bp, Artificial Sequence
  Description of Artificial Sequence Synthetic polynucleotide
gaaaatgttctcacccagtctccagcaatcatgtctgcatctccaggggaaaaggtcaccatgacctgcagtgccaggtcaagtgtaagttacatgcactggtaccagcagaagtcaaccgcctcccccaaactctggatttatgacacatccaaactggcttctggagtcccaggtcgcttcagtggcagtgggtctggaaactcttactctctcacgatcagcagcatggaggctgaagatgttgccacttattactgttttcaggcgagtgggtacccgctcacgttcggtgctgggaccaagctggagctgaaacgg

SEQ ID NO: 3 - protein, 117 aa, Artificial Sequence
  Description of Artificial Sequence Synthetic polypeptide
QVQLKESGPGLVAPSQSLSITCTVSGFSLISYGVDWVRQPPGKGLEWLGVIWGGGNTNYNSSLMSRLSISKDNSKSQVFLKMNSLQTDDTAMYYCAKTGTGYALEYWGQGTSVTVSS

SEQ ID NO: 4 - protein, 107 aa, Artificial Sequence
  Description of Artificial Sequence Synthetic polypeptide
ENVLTQSPAIMSASPGEKVTMTCSARSSVSYMHWYQQKSTASPKLWIYDTSKLASGVPGRFSGSGSGNSYSLTISSMEAEDVATYYCFQASGYPLTFGAGTKLELKR

SEQ ID NO: 5 - protein, 23 aa, Artificial Sequence
  Description of Artificial Sequence Synthetic peptide
ENVLTQSPAIMSASPGEKVTMTC

SEQ ID NO: 6 - protein, 10 aa, Artificial Sequence
  Description of Artificial Sequence Synthetic peptide
SARSSVSYMH

SEQ ID NO: 7 - protein, 15 aa, Artificial Sequence
  Description of Artificial Sequence Synthetic peptide
WYQQKSTASPKLWIY

SEQ ID NO: 8 - protein, 7 aa, Artificial Sequence
  Description of Artificial Sequence Synthetic peptide
DTSKLAS

SEQ ID NO: 9 - protein, 32 aa, Artificial Sequence
  Description of Artificial Sequence Synthetic polypeptide
GVPGRFSGSGSGNSYSLTISSMEAEDVATYYC

SEQ ID NO: 10 - protein, 9 aa, Artificial Sequence
  Description of Artificial Sequence Synthetic peptide
FQASGYPLT

SEQ ID NO: 11 - protein, 11 aa, Artificial Sequence
  Description of Artificial Sequence Synthetic peptide
FGAGTKLELKR

SEQ ID NO: 12 - protein, 25 aa, Artificial Sequence
  Description of Artificial Sequence Synthetic peptide
QVQLKESGPGLVAPSQSLSITCTVS

SEQ ID NO: 13 - protein, 10 aa, Artificial Sequence
  Description of Artificial Sequence Synthetic peptide
GFSLISYGVD

SEQ ID NO: 14 - protein, 14 aa, Artificial Sequence
  Description of Artificial Sequence Synthetic peptide
WVRQPPGKGLEWLG

SEQ ID NO: 15 - protein, 16 aa, Artificial Sequence
  Description of Artificial Sequence Synthetic peptide
VIWGGGNTNYNSSLMS

SEQ ID NO: 16 - protein, 32 aa, Artificial Sequence
  Description of Artificial Sequence Synthetic polypeptide
RLSISKDNSKSQVFLKMNSLQTDDTAMYYCAK

SEQ ID NO: 17 - protein, 9 aa, Artificial Sequence
  Description of Artificial Sequence Synthetic peptide
TGTGYALEY

SEQ ID NO: 18 - protein, 11 aa, Artificial Sequence
  Description of Artificial Sequence Synthetic peptide
WGQGTSVTVSS

SEQ ID NO: 19 - DNA, 351 bp, Artificial Sequence
  Description of Artificial Sequence Synthetic polynucleotide
caggtgcagctgaaggagtcaggacctggcctggtggcgccctcacagagcctgtccatcacttgtactgtctctgggttttcattaagcagctatggtgtagactgggttcgccaacctccaggaaagggtctggagtggctgggagtaatatggggtggtggaagcataaattataattcagctctcatgtccagactgagcatcagcaaagacaattccaagagccaaattttcttaaaaatgaacagtctgcaaactgatgacacagccatatactactgtaccacacatgaggattacggtccttttgcttactggggccaagggactctggtcactgtctctgca

SEQ ID NO: 20 - DNA, 321 bp, Artificial Sequence
  Description of Artificial Sequence Synthetic polynucleotide
caaattgttctctcccagtctccagcaatcctgtctgcatctccaggggagaaggtcacaatgacttgcagggccagctcaagtgtaagttacatgcactggtaccagcagaagccaggatcctcccccaaatcctggatttatgccacatccaacctggcttctggagtccctgctcgcttcagtggcagtgggtctgggacctcttactctctcacaatcagcagagtggaggctgaagatgctgccacttattactgccagcagtggggtagttacccgtggacgttcggtggaggcaccaagctggaaatcaaacgg

SEQ ID NO: 21 - protein, 117 aa, Artificial Sequence
  Description of Artificial Sequence Synthetic polypeptide
QVQLKESGPGLVAPSQSLSITCTVSGFSLSSYGVDWVRQPPGKGLEWLGVIWGGGSINYNSALMSRLSISKDNSKSQIFLKMNSLQTDDTAIYYCTTHEDYGPFAYWGQGTLVTVSA

SEQ ID NO: 22 - protein, 107 aa, Artificial Sequence
  Description of Artificial Sequence Synthetic polypeptide
QIVLSQSPAILSASPGEKVTMTCRASSSVSYMHWYQQKPGSSPKSWIYATSNLASGVPARFSGSGSGTSYSLTISRVEAEDAATYYCQQWGSYPWTFGGGTKLEIKR

SEQ ID NO: 23 - protein, 23 aa, Artificial Sequence
  Description of Artificial Sequence Synthetic peptide
QIVLSQSPAILSASPGEKVTMTC

SEQ ID NO: 24 - protein, 10 aa, Artificial Sequence
  Description of Artificial Sequence Synthetic peptide
RASSSVSYMH

SEQ ID NO: 25 - protein, 15 aa, Artificial Sequence
  Description of Artificial Sequence Synthetic peptide
WYQQKPGSSPKSWIY

SEQ ID NO: 26 - protein, 7 aa, Artificial Sequence
  Description of Artificial Sequence Synthetic peptide
ATSNLAS

SEQ ID NO: 27 - protein, 32 aa, Artificial Sequence
  Description of Artificial Sequence Synthetic polypeptide
GVPARFSGSGSGTSYSLTISRVEAEDAATYYC

SEQ ID NO: 28 - protein, 9 aa, Artificial Sequence
  Description of Artificial Sequence Synthetic peptide
QQWGSYPWT

SEQ ID NO: 29 - protein, 11 aa, Artificial Sequence
  Description of Artificial Sequence Synthetic peptide
FGGGTKLEIKR

SEQ ID NO: 30 - protein, 25 aa, Artificial Sequence
  Description of Artificial Sequence Synthetic peptide
QVQLKESGPGLVAPSQSLSITCTVS

SEQ ID NO: 31 - protein, 10 aa, Artificial Sequence
  Description of Artificial Sequence Synthetic peptide
GFSLSSYGVD

SEQ ID NO: 32 - protein, 14 aa, Artificial Sequence
  Description of Artificial Sequence Synthetic peptide
WVRQPPGKGLEWLG

SEQ ID NO: 33 - protein, 16 aa, Artificial Sequence
  Description of Artificial Sequence Synthetic peptide
VIWGGGSINYNSALMS

SEQ ID NO: 34 - protein, 32 aa, Artificial Sequence
  Description of Artificial Sequence Synthetic polypeptide
RLSISKDNSKSQIFLKMNSLQTDDTAIYYCTT

SEQ ID NO: 35 - protein, 9 aa, Artificial Sequence
  Description of Artificial Sequence Synthetic peptide
HEDYGPFAY

SEQ ID NO: 36 - protein, 11 aa, Artificial Sequence
  Description of Artificial Sequence Synthetic peptide
WGQGTLVTVSA

SEQ ID NO: 37 - DNA, 351 bp, Artificial Sequence
  Description of Artificial Sequence Synthetic polynucleotide
caggtgcagctgaaggagtcaggacctggcctggtggcgccctcacagagcctgtccatcacatgcactgtctcagggttttcattaaccagttatggtgtaagctgggctcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtgccaaaccggaaaactgggacggcttcgatgtctggggcccagggaccacggtcaccgtctcctca

SEQ ID NO: 38 - DNA, 321 bp, Artificial Sequence
  Description of Artificial Sequence Synthetic polynucleotide
caaattgttctctcccagtctccagcaatcctgtctgcatctccaggggagaaggtcacaatgacttgcagggccagctcaagtgtaagttacatgcactggtaccgacagaagccaggatcctcccccaaaccctggatttatgccacatccgacctggcttctggagtccctactcgcttcagtggcagtgggtctgggacctcttactctctcacaatcagcagagtggaggctgaagatgctgccacttattactgccagcagtggagtagttacccgtggacgttcggtggaggcaccaagctggaaatcaaacgg

SEQ ID NO: 39 - protein, 117 aa, Artificial Sequence
  Description of Artificial Sequence Synthetic polypeptide
QVQLKESGPGLVAPSQSLSITCTVSGFSLTSYGVSWARQPPGKGLEWLGVIWGDGSTNYHSALISRLSISKDNSKSQVFLKLNSLQTDDTATYYCAKPENWDGFDVWGPGTTVTVSS

SEQ ID NO: 40 - protein, 107 aa, Artificial Sequence
  Description of Artificial Sequence Synthetic polypeptide
QIVLSQSPAILSASPGEKVTMTCRASSSVSYMHWYRQKPGSSPKPWIYATSDLASGVPTRFSGSGSGTSYSLTISRVEAEDAATYYCQQWSSYPWTFGGGTKLEIKR

SEQ ID NO: 41 - protein, 23 aa, Artificial Sequence
  Description of Artificial Sequence Synthetic peptide
QIVLSQSPAILSASPGEKVTMTC

SEQ ID NO: 42 - protein, 10 aa, Artificial Sequence
  Description of Artificial Sequence Synthetic peptide
RASSSVSYMH

SEQ ID NO: 43 - protein, 15 aa, Artificial Sequence
  Description of Artificial Sequence Synthetic peptide
WYRQKPGSSPKPWIY

SEQ ID NO: 44 - protein, 7 aa, Artificial Sequence
  Description of Artificial Sequence Synthetic peptide
ATSDLAS

SEQ ID NO: 45 - protein, 31 aa, Artificial Sequence
  Description of Artificial Sequence Synthetic polypeptide
VPTRFSGSGSGTSYSLTISRVEAEDAATYYC

SEQ ID NO: 46 - protein, 9 aa, Artificial Sequence
  Description of Artificial Sequence Synthetic peptide
QQWSSYPWT

SEQ ID NO: 47 - protein, 11 aa, Artificial Sequence
  Description of Artificial Sequence Synthetic peptide
FGGGTKLEIKR

SEQ ID NO: 48 - protein, 25 aa, Artificial Sequence
  Description of Artificial Sequence Synthetic peptide
QVQLKESGPGLVAPSQSLSITCTVS

SEQ ID NO: 49 - protein, 10 aa, Artificial Sequence
  Description of Artificial Sequence Synthetic peptide
GFSLTSYGVS

SEQ ID NO: 50 - protein, 14 aa, Artificial Sequence
  Description of Artificial Sequence Synthetic peptide
WARQPPGKGLEWLG

SEQ ID NO: 51 - protein, 16 aa, Artificial Sequence
  Description of Artificial Sequence Synthetic peptide
VIWGDGSTNYHSALIS

SEQ ID NO: 52 - protein, 32 aa, Artificial Sequence
  Description of Artificial Sequence Synthetic polypeptide
RLSISKDNSKSQVFLKLNSLQTDDTATYYCAK

SEQ ID NO: 53 - protein, 9 aa, Artificial Sequence
  Description of Artificial Sequence Synthetic peptide
PENWDGFDV

SEQ ID NO: 54 - protein, 11 aa, Artificial Sequence
  Description of Artificial Sequence Synthetic peptide
WGPGTTVTVSS

SEQ ID NO: 55 - protein, 117 aa, Artificial Sequence
  Description of Artificial Sequence Synthetic polypeptide
QVQLQESGPGLVKPSQTLSLTCTVSGFSLSSYGVDWVRQPPGKGLEWVGVIWGGGNTNYNSSLMSRFTISRDNSKNTLYLQMNSLKTEDTAVYYCAKTGTGYALEYWGQGTTVTVSS

SEQ ID NO: 56 - protein, 117 aa, Artificial Sequence
  Description of Artificial Sequence Synthetic polypeptide
QVKLKESGPGLVKPTQTLTLTCTVSGFSLSSYGVDWVRQPPGKGLEWVGVIWGGGNTNYNSSLMSRFTISRDNSKNTLYLQMNSLKTEDTAVYYCAKTGTGYALEYWGQGTTVTVSS

SEQ ID NO: 57 - protein, 117 aa, Artificial Sequence
  Description of Artificial Sequence Synthetic polypeptide
QVKLKESGPGLVKPTQTLTLTCTVSGFSLSSYGVDWVRQPPGKGLEWVGVIWGGGSTNYNSSLMSRFTISRDNSKNTLYLQMNSLKTEDTAVYYCAKTGTGYALEYWGQGTTVTVSS

SEQ ID NO: 58 - protein, 117 aa, Artificial Sequence
  Description of Artificial Sequence Synthetic polypeptide
QVKLKESGPGLVKPTQTLTLTCTVSGFSLSSYGVDWVRQPPGKGLEWVGVIWGGGQTNYNSSLMSRFTISRDNSKNTLYLQMNSLKTEDTAVYYCAKTGTGYALEYWGQGTTVTVSS

SEQ ID NO: 59 - protein, 117 aa, Artificial Sequence
  Description of Artificial Sequence Synthetic polypeptide
QVKLKESGPGLVKPTQTLTLTCTVSGFSLSSYGVDWVRQPPGKGLEWVGVIWGGGNTYYNSSLMSRFTISRDNSKNTLYLQMNSLKTEDTAVYYCAKTGTGYALEYWGQGTTVTVSS

SEQ ID NO: 60 - protein, 117 aa, Artificial Sequence
  Description of Artificial Sequence Synthetic polypeptide
QVTLKESGPGLVKPTQTLTLTCTVSGFSLSSYGVDWVRQPPGKGLEWVGVIWGGGSTNYNSSLMSRFTISRDNSKNTLYLQMNSLKTEDTAVYYCAKTGTGYALEYWGQGTTVTVSS

SEQ ID NO: 61 - protein, 117 aa, Artificial Sequence
  Description of Artificial Sequence Synthetic polypeptide
QVTLKESGPGLVKPTQTLTLTCTVSGFSLSSYGVDWVRQPPGKGLEWVGVIWGGGQTNYNSSLMSRFTISRDNSKNTLYLQMNSLKTEDTAVYYCAKTGTGYALEYWGQGTTVTVSS

SEQ ID NO: 62 - protein, 117 aa, Artificial Sequence
  Description of Artificial Sequence Synthetic polypeptide
QVTLKESGPGLVKPTQTLTLTCTVSGFSLSSYGVDWVRQPPGKGLEWVGVIWGGGNTYYNSSLMSRFTISRDNSKNTLYLQMNSLKTEDTAVYYCAKTGTGYALEYWGQGTTVTVSS

SEQ ID NO: 63 - protein, 117 aa, Artificial Sequence
  Description of Artificial Sequence Synthetic polypeptide
QVTLKESGPALVKPTQTLTLTCTVSGFSLSSYGVDWVRQPPGKGLEWVGVIWGGGNTNYNSSLMSRFTISRDNSKNTLYLQMNSLKTEDTAVYYCAKTGTGYALEYWGQGTTVTVSS

SEQ ID NO: 64 - protein, 117 aa, Artificial Sequence
  Description of Artificial Sequence Synthetic polypeptide
QVKLKESGPALVKPSQTLTLTCTVSGFSLSSYGVDWVRQPPGKGLEWVGVIWGGGNTNYNSSLMSRFTISRDNSKNTLYLQMNSLKTEDTAVYYCAKTGTGYALEYWGQGTTVTVSS

SEQ ID NO: 65 - protein, 117 aa, Artificial Sequence
  Description of Artificial Sequence Synthetic polypeptide
QVKLKESGPGLVKPSQTLTLTCTVSGFSLSSYGVDWVRQPPGKGLEWVGVIWGGGNTNYNSSLMSRFTISRDNSKNTLYLQMNSLKTEDTAVYYCAKTGTGYALEYWGQGTTVTVSS

SEQ ID NO: 66 - protein, 117 aa, Artificial Sequence
  Description of Artificial Sequence Synthetic polypeptide
QVKLQESGPALVKPSQTLTLTCTVSGFSLSSYGVDWVRQPPGKGLEWVGVIWGGGNTNYNSSLMSRFTISRDNSKNTLYLQMNSLKTEDTAVYYCAKTGTGYALEYWGQGTTVTVSS

SEQ ID NO: 67 - protein, 11 aa, Artificial Sequence
  Description of Artificial Sequence Synthetic peptide
GFSLSSYGVDW

SEQ ID NO: 68 - protein, 17 aa, Artificial Sequence
  Description of Artificial Sequence Synthetic peptide
VIWGGGNTNYNSSLMSR

SEQ ID NO: 69 - protein, 8 aa, Artificial Sequence
  Description of Artificial Sequence Synthetic peptide
TGTGYALE

SEQ ID NO: 70 - protein, 107 aa, Artificial Sequence
  Description of Artificial Sequence Synthetic polypeptide
DIQMTQSPSSLSASVGDRVTITCSARSSVSYMHWYQQKPGKVPKLLIYDTSKLASGVPSRFSGSGSGTDFTLTISSLQPEDVATYYCFQASGYPLTFGGGTKVEIKR

SEQ ID NO: 71 - protein, 107 aa, Artificial Sequence
  Description of Artificial Sequence Synthetic polypeptide
EIVLTQSPATLSLSPGERATLSCSARSSVSYMHWYQQKPGQAPRLLIYDTSKLASGIPARFSGSGSGTDFTLTISSLEPEDFAVYYCFQASGYPLTFGGGTKVEIKR

SEQ ID NO: 72 - protein, 10 aa, Artificial Sequence
  Description of Artificial Sequence Synthetic peptide
SARSSVSYMH

SEQ ID NO: 73 - protein, 7 aa, Artificial Sequence
  Description of Artificial Sequence Synthetic peptide
DTSKLAS

SEQ ID NO: 74 - protein, 9 aa, Artificial Sequence
  Description of Artificial Sequence Synthetic peptide
FQASGYPLT

(19) United States
(12) Patent Application Publication
(10) Pub. No.: US 2020/0138967 A1
(43) Pub. Date: May 7, 2020

(54) COMBINATION THERAPY USING ANTI-SSEA-4 ANTIBODY IN COMBINATION WITH THERAPEUTIC ONCOLOGY AGENTS

(71) Applicant: OBI PHARMA, INC., Taipei City (TW)

(72) Inventors: Cheng-Der Tony YU, San Diego, CA (US); Jiann-Shiun LAI, (US); Yi-Chien TSAI, Taipei City (TW)

(21) Appl. No.: 16/590,967

(22) Filed: Oct. 2, 2019

Related U.S. Application Data
(60) Provisional application No. 62/740,373, filed on Oct. 2, 2018.

(51) Int. Cl. A61K 47/64; A61K 47/68; C07K 16/28; A61P 35/00; A61P 31/00

(52) U.S. Cl. CPC A61K 47/6425; A61K 47/6853; A61P 31/00; A61P 35/00; C07K 16/2815

(57) ABSTRACT

The present disclosure is generally directed to treatment methods and compositions comprising administering anti-SSEA-4 antibodies; alone or in additive and/or synergistic combination with other therapeutic agents in oncology to enhance therapeutic efficacy whereby the interaction alters the epitope binding of Siglec-9 protein; including human Siglec-9 or a mammalian Siglec-9; wherein the use of such anti-SSEA-4 compositions are efficacious in preventing, reducing risk, or treating an individual with cancer.

This application claims the benefit of U.S. Patent Application No. 62/740,373, filed on Oct. 2, 2018, the disclosure of which is incorporated by reference herein in its entirety.

### FIELD

The present disclosure is generally directed to treatment methods and compositions comprising anti-SSEA-4 antibodies, e.g., monoclonal, chimeric, humanized antibodies, antibody fragments, etc.; either alone or in combination with other therapeutic oncology agents. Said methods and compositions can synergistically modulate binding of Siglec-9 protein, e.g., human Siglec-9 or a mammalian Siglec-9, and the use of said treatment methods and compositions is useful in preventing, reducing risk, or treating an individual with cancer.

### BACKGROUND OF THE INVENTION

SSEA-4 (stage-specific embryonic antigen-4), a hexasaccharide (Neu5Acα2-3Galβ1-3GalNAcβ1-3Galα1-4Galβ1-4Glcβ), is commonly used as a cell surface marker for pluripotent human embryonic stem cells; It is also used to isolate mesenchymal stem cells and enrich neural progenitor cells (Kannagi et al. (1983) EMBO J. 2: 2355-236) Recent studies show that SSEA-4 is implicated in the malignancy of cancers, such as invasion and metastasis of cancer cells (Kavitha et al. (2015 Glycobiology 25: 902-917; Lou et al. (2014) Proc Natl Acad Sci USA. 111: 2482-2487).

The expression of SSEA-4 is associated with the increase in metastatic potential and poor prognosis of lung, renal, breast and oral cancer (Kataguri et al. (2001) Glycoconj J. 18:347-353; Gottschling et al. (2013) Eur Respir J. 41:656-663; Hung et al. (2013) J Am Chem Soc. 135:5934-5937). However, in normal tissues, SSEA-4 has been reported to be expressed as minor GSLs in erythrocytes and on the epithelial cells of several glandular tissues (Kannagi et al. (1983) EMBO J. 2: 2355-236). Due to its property, SSEA-4 may serve as a promising unique target for cancer immunotherapy.

Siglecs (Sialic acid-binding immunoglobulin-type lectins) is a family of cell surface proteins that bind sialic acid and found primarily on immune cells. There are 14 different mammalian Siglecs, providing an array of different functions based on cell surface receptor-ligand interactions (Pillai et al. (2012) Annual Review of Immunology. 30: 357-92). Most Siglecs inhibit immune cell activation due to their ITIM-containing cytoplasmic regions. Siglecs recruit inhibitory proteins such as SHP phosphatases via their ITIM domains when bound to their ligand (Avril et al. (2004) Journal of Immunology. 173 (11): 6841-9) which lead to inactivate the immune cells. Thus, disrupt the interaction between Siglecs and their ligands may promote anti-tumor immunity.

Sialic acid-binding immunoglobulin (Ig)-like lectins, or SIGLECs (e.g., CD33), are a family of type 1 transmembrane proteins, each having a unique expression pattern, mostly in hemopoietic cells. The CD33-like subgroup of SIGLECs, which are localized to 19q13.3-q13.4, have 2 conserved cytoplasmic tyrosine-based motifs: an immunoreceptor tyrosine-based inhibitory motif, or ITIM and a motif homologous to one identified in signaling lymphocyte activation molecule (SLAM) that mediates an association with SLAM-associated protein (SAP).

Sialic acid-binding Ig-like lectin-9 (Siglec-9), is a type 1, immunoglobulin-like, transmembrane protein expressed on immune and hematopoietic cells, including immature and mature myeloid cells, such as monocytes, macrophages, dendritic cells, neutrophils, and microglial cells, as well as lymphoid cells, such as natural killer cells, B cells and subsets of CD8⁺ T cells (Crocker et al. (2007) Nat Rev Immunol. 7:255-266; O'Reilly and Paulson (2009) Trends in Pharm. Sci. 30:5:240-248; and Macauley et al. (2014) Nat. Rev. Imm. 14: 653-666). Siglec-9 is a member of the Siglec family of lectins that bind sialic acid residues of glycoproteins and glycolipids. One potential binding target for Siglec proteins is gangliosides; that is, glycolipids that consist of a ceramide linked to a sialylated glycan. Most gangliosides share a common lacto-ceramide core and one or more sialic acid residues. Diversity in the Siglec ligands is generated by the addition of other neutral sugars and sialic acid in different linkages, either branched or terminal, and modification of sialic acid itself.

Fourteen Siglec proteins have been identified in humans and nine in mice that are comprised of 2-17 extracellular Ig domains including an amino-terminal V-set domain that contains the sialic acid-binding site. The sialic acid-binding region is located on the V-set Ig-like domain, which contains a two aromatic residues and one arginine motif highly conserved in all Siglecs (Crocker et al. (2007) Nat Rev Immunol. 7:255-266; McMillan and Crocker (2008) Carbohydr Res. 343:2050-2056; Von Gunten and Bochner (2008) Ann NY Acad Sci. 1143:61-82; May et al. (1998) Mol Cell. 1:719-728; Crocker et al. (1999) Biochem J. 341:355-361; and Crocker and Varki (2001) Trends Immunol. 2:337-342). The binding sites to sialylated ligands have been mapped by crystal structures with and without ligand bound (Attrill et al. (2006) J. Biol. Chem. 281 32774-32783; Alphey et al. (2003) J. Biol. Chem. 278:5 3372-3377; Varki et al., Glycobiology, 16 pp. 1R-27R; and May et al. (1998) Mol. Cell 1:5:719-728). Since cell membranes are rich in sialic acids, ligand binding by Siglecs can occur in cis and in trans, both affecting their functional properties. Each Siglec has a distinct preference for binding the diverse types of sialylated glycans that are found on the surface of mammalian cells (Crocker et al. (2007) Nat Rev Immunol. 7:255-266; and Crocker et al. (2007) Nat Rev Immunol. 7:255-266). Most Siglec proteins, including Siglec-9, contain one or more immunoreceptor tyrosine-based inhibitory motif (ITIM) sequences in their cytoplasmic tails, which enable them as inhibitory receptors and negative regulators of immune functions through recruitment of the tyrosine phosphatases SHP1 and SHP2 (Crocker et al. (2007) Nat Rev Immunol. 7:255-266; McMillan and Crocker (2008) Carbohydr Res. 343:2050-2056; and Von Gunten and Bochner (2008) Ann NY Acad Sci. 1143:61-82). Certain Siglecs contain immunoreceptor tyrosine-based activating motif (ITAM) sequences in their cytoplasmic tails, which enable them to act as activating receptors and positive regulators of immune function through predicted recruitment of spleen tyrosine kinase (Syk) (Macauley S M. et al. (2014) Nature Reviews Immunology 14, 653-666). The Siglec protein family is associated with multiple human diseases including, autoimmunity, susceptibility to infection, multiple types of cancer including lymphoma, leukemia and acute myeloid leukemia, systemic lupus erythematosus, rheumatoid arthritis, neurodegenerative disorders, asthma, allergy, sepsis, chronic obstructive pulmonary disease, graft-versus-host disease, eosinophilia, and osteoporosis (Macauley S M. et al. (2014) Nature Reviews Immunology 14, 653-666).

Siglec-9 was cloned in 2000 from peripheral blood mononuclear cells (Angata and Varki (2000) J. Biol. Chem. 275:29: 22127-22135) and selective expression was detected on granulocytes and monocytes. An independent group isolated Siglec-9 from HL-60 (human promyelocytic leukemia) cells and demonstrated expression on monocytes, neutrophils, NK cells, B cells and a small subset of CD8⁺ T cells (Zhang et al. (2000) J. Biol. Chem. 275:29 22121-22126).

Siglec-9 contains an extracellular N-terminal Ig-like (immunoglobulin-like) V-type domain, two Ig-like C2-set domains as well as two consensus ITIM motifs in its cytoplasmic domain. Expression of Siglec-9 in COS cells demonstrated sialic acid-dependent binding of red blood cells, which is mediated by terminal a2-3 or a2-6 sialic acid linkages (Angata and Varki (2000) J. Biol. Chem. 275: 22127-22135, Zhang et al. (2000) J. Biol. Chem. 275:29 22121-22126). It was further confirmed that Siglec-9 is masked by endogenous cellular sialic acids and binds to exogenous terminal a2-3 or a2-6 sialic acid probes only upon sialidase treatment of the cells (Yamaji (2002) J. Biol. Chem. 277:8 6324-6332). Ligand specificity within the N-terminal V-set Ig-like domain of Siglec-9 was mapped to a small region, Asn70-Lys75, by swapping Siglec-7 with Siglec-9 regions and vice versa. Acquisition of the respective Siglec ligand specificity within these amino acid residues supports the notion that ligand specificity is dictated by interactions in the variable C—C′ loop (Yamaji (2002) J. Biol. Chem. 277:8 6324-6332). Pathogens have apparently subverted the sialic acid as “self’ system as it has been reported that group B Streptococcus can bind Siglec-9 on human neutrophils thereby reducing the immune response to the bacteria, which can either be pathogenic or commensal (Carlin et al (2009) Blood 113: 3333-3336). Other sources of in vivo Siglec-9 sialic acid ligands are tumor-secreted mucins, such as MUC1, MUC2, MUC16; Siglec-9 was shown to bind mucins from the sera of cancer patients (Ohta et al. (2010) Biochem. and Biophys. Res. Comm. 402: 663-669; Belisle et al. (2010) Mol. Cancer 9:118).

Siglec-9 undergoes phosphorylation of Tyr-433, and Tyr-456 by tyrosine kinases, likely c-Src or Lck, and functions as an inhibitory receptor (Avril et al. (2004) J. Imm. 173: 6841-6849). Following phosphorylation on the proximal Tyr-433 in the ITIM domain, Siglec-9 binds SHP-2/PTPN11 and SHP-1/PTPN6. The membrane distal ITIM motif does not appear to contribute significantly as mutation did not preclude tyrosine phosphorylation or inhibitory function of Siglec-9. Siglec-9 was shown to inhibit FcERI-mediated activities in rat basophilic leukemia cells, which have been previously used to characterize an inhibitory receptor class expressed on NK cells called KIRs (Killer Ig-like receptors) (Avril et al. (2004) J. Imm. 173: 6841-6849).

Phosphatase activity is additionally associated with decreased intracellular calcium mobilization, and decreased tyrosine phosphorylation on multiple proteins (Ulyanova, T., et al. (1999) Eur J Immunol 29, 3440-3449; Paul, S. P., et al. (2000). Blood 96, 483-490) as well as with blockade of signal transduction and immune response, in part, through dephosphorylation of signaling molecules on adjacent activating receptors, including those that contain ITAM motifs, pattern recognition receptors, Toll-like receptors and damage-associated molecular pattern (DAMP) receptors. It has been proposed that the association between ITIM-containing Siglec receptors and activating receptors may be mediated by extracellular ligands that bind and bridge these receptors (Macauley S M. et al. (2014) Nature Reviews Immunology 14, 653-666). Some, but not all, Siglec ligands induce receptor downregulation (Macauley S M. et al. (2014) Nature Reviews Immunology 14, 653-666). Ligand-induced receptor degradation has been reported for tyrosine kinase receptors (Monsonego-Oran et al. (2002) Febs letters 528, 83-89; and Fasen et al. (2008) Cell & Molecular Biology 9. 251-266), as well as for steroid receptors (Callige et al. (2005) Mol. Cell. Biol. 25. 4349-4358; and Pollenz et al. (2006) Chemico-Biological Interactions. 164. 49-59). Siglec-9 is thought to be to constitutively recycled in acute myeloid leukemia (AML) cells and has been shown to mediate rapid endocytosis of an anti-Siglec-9 monoclonal antibody on these cells (Biedermann et al. (2007) Leuk. Res. 31:2:211-220). However, no decrease in cellular levels of Siglec-9 has been reported in either AML or normal primary immune cells. Likewise, no receptor recycling or antibody-dependent receptor down regulation has been reported in any type of primary cells. Expression of Siglec-9 on the cell surface is dependent in part on the membrane proximal ITIM motif, but not the distal motif, according to mutational analysis performed in an overexpression system (Biedermann et al. (2007) Leuk. Res. 31:2:211-220).

### SUMMARY OF THE INVENTION

The present disclosure is based on the surprising discovery that stage-specific embryonic antigen 4 (SSEA-4) can selectively bind Siglec-9 and thereby modulate tumor presentation to immune cells. Specifically, the present disclosure provides methods and compositions comprising SSEA-4 antibodies for modulating SSEA-4-Siglec-9 interaction whereby tumor immune presentation is enhanced and NK-cell mediated cytotoxicity is improved.

In one aspect, the present disclosure features an antibody or binding fragment thereof specific to SSEA-4 that can modulate SSEA-4 and Siglec-9 binding. The anti-SSEA-4 antibody binds to Neu5Acα2→3Galβ1→3GalNAcrβ1→3Galα1→4Galβ1→4Glcβ1.

In one aspect, the present disclosure provides a method of treating a subject having a tumor cell expressing a SSEA-4 antigen, the method comprising administering to the subject an effective amount of a pharmaceutical composition comprising an anti-SSEA-4 antibody or a fragment thereof.

In one embodiment, the binding of anti-SSEA-4 antibody to the tumor cell decreases the binding interaction between SSEA-4 and Siglec-9.

In one embodiment, the decrease in the binding interaction between SSEA-4 and Siglec-9 results in the decrease binding of Siglec-9 to tumor cells. In one embodiment, the decrease binding of Siglec-9 to tumor cells induces a release of the immunosuppression (immune-masking) maintained by Siglec-9/SSEA-4 engagement.

In one embodiment, the administering of the anti-SSEA-4 antibody increases the activity of cytotoxic immune cells. In one embodiment, the cytotoxic immune cell is an NK cell.

In certain embodiments, the antibody or antigen-binding fragment thereof is selected from: (a) a whole immunoglobulin molecule;

(b) an scFv;
(c) a Fab fragment;

### (d) an F(ab′)₂; or

(e) a disulfide linked Fv.

In certain embodiments, the antibody is a humanized antibody.

In certain embodiments, the antibody is an IgG or IgM.

In certain embodiments, the pharmaceutical composition further comprises at least one additional therapeutic agent.

In one aspect, the present disclosure provides a method for inhibiting the proliferation of cancer cells, comprising the administering of an effective amount of an exemplary pharmaceutical composition to a subject in need thereof, wherein the proliferation of cancer cells is inhibited. Cancers expressing SSEA-4 include, but are not limited to, sarcoma, leukemia, lymphoma, glioblastoma, lung cancer, breast cancer, lung cancer, esophageal cancer, colorectal cancer, biliary cancer, liver cancer, buccal cancer, gastric cancer, colon cancer, nasopharyngeal cancer, oropharyngeal cancer, laryngeal cancer, esophageal cancer, stomach cancer, liver cancer, bile duct cancer, gallbladder cancer, bladder cancer, intestinal cancer, kidney cancer, prostate cancer, ovarian cancer, cervical cancer, endometrial cancer, pancreatic cancer, testicular cancer, bladder cancer, head and neck cancer, oral cancer, neuroendocrine cancer, adrenal cancer, thyroid cancer, bone cancer, skin cancer, basal cell carcinoma, squamous cell carcinoma, melanoma, or brain tumor.

In certain embodiments, the present disclosure provides a method of treating cancer in a subject. The method comprises administering to a subject in need thereof an effective amount of the exemplary antibody described herein.

The details of one or more embodiments of the invention are set forth in the description below. Other features and/or advantages of the present invention will be apparent from the drawings, detailed description of several embodiments, and also from the appending claims.

### BRIEF DESCRIPTION OF THE FIGURES

A more complete understanding of the invention may be obtained by reference to the accompanying drawings, when considered in conjunction with the subsequent detailed description. The embodiments illustrated in the drawings are intended only to exemplify the invention and should not be construed as limiting the invention to the illustrated embodiments.

FIG. 1. ELISA binding assay of Siglec-9 and SSEA-4 ceramide.

FIG. 2. Increase the Siglec-9 binding on lung cancer cell line (A549) by exogeneous of SSEA-4 ceramide.

FIG. 3. Reduction of the Siglec-9 binding on breast cancer cell line (MDA-MB 231) by the addition of exemplary anti-SSEA-4 Fab (OBI-898).

FIG. 4. Binding of SSEA-4 by anti-SSEA-4 Fab (OBI-898) on breast cancer cell line (MDA-MB 231) enhanced the cellular cytotoxicity of human PBMCs.

FIG. 5. Binding of SSEA-4 by anti-SSEA-4 Fab (OBI-898) on ovary cancer cell line (SKOV-3) enhanced the cellular cytotoxicity of human PBMCs.

FIG. 6. Binding of SSEA-4 by anti-SSEA-4 Fab (OBI-898) on ovary cancer cell line (SKOV-3) enhanced the cellular cytotoxicity of Tecentriq.

FIG. 7. Schematic of the mechanism of action of anti-SSEA-4 antibody (OBI-898) with Siglec-9 to release the immunosuppression.

FIG. 8. The structure of stage-specific embryonic antigen 4 (SSEA-4)

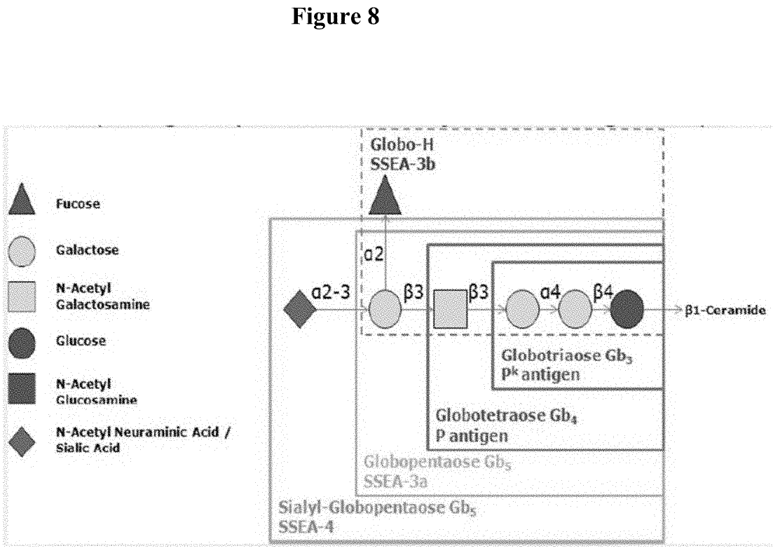

FIG. 9. Glycan binding specificities of human Sialic acid-binding immunoglobulin-type lectins (Siglecs)

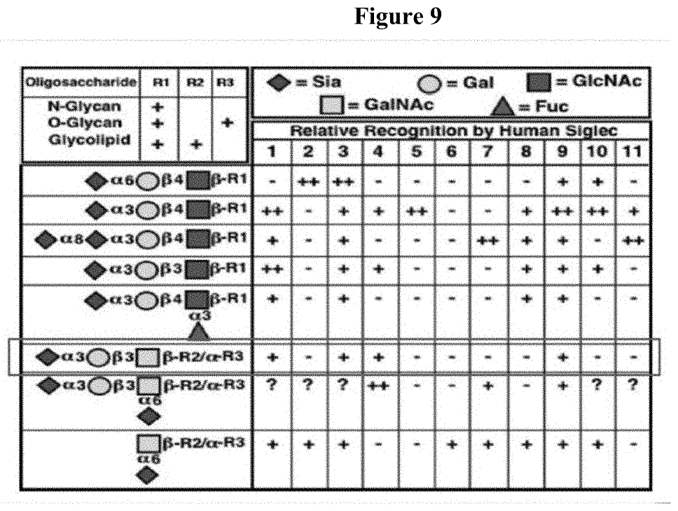

FIG. 10. The exemplary ligands of Sialic acid-binding Ig-like lectin-9 (Siglec-9).

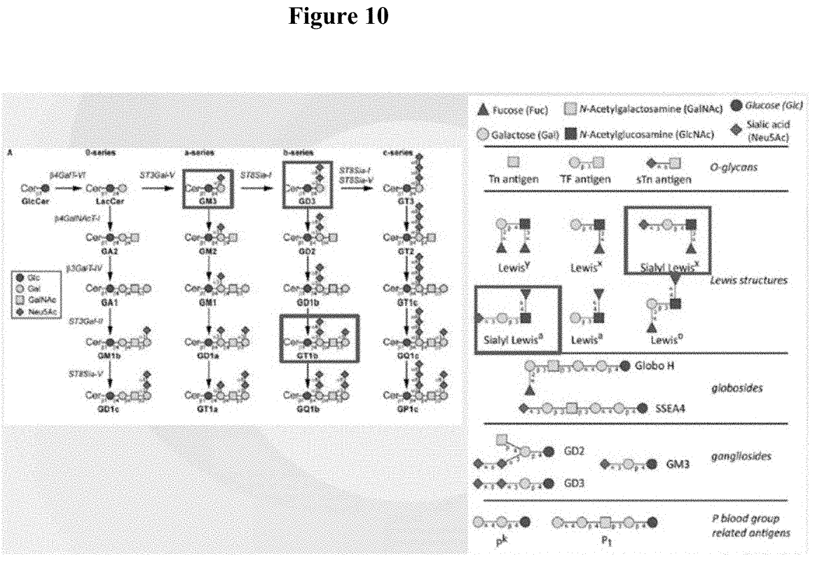

### DETAILED DESCRIPTION OF THE INVENTION

The present disclosure is based on the surprising discovery that stage-specific embryonic antigen 4 (SSEA-4) can selectively bind Siglec-9 and thereby modulate tumor presentation to immune cells. Specifically, the present disclosure provides methods and compositions comprising SSEA-4 antibodies for modulating SSEA-4-Siglec-9 interaction whereby tumor immune presentation is enhanced and NK-cell mediated cytotoxicity is improved.

The present disclosure describes the use of anti-SSEA-4 antibody as a new immune checkpoint blockade therapy in cancer immunotherapy. SSEA-4 was first proved to be a new tumor associated carbohydrate ligand for Siglec-9. Anti-SSEA-4 antibody were discovered to block the interaction between Siglec-9 and SSEA-4. In vitro assays showed that anti-SSEA-4 antibody potently reverse inhibitory functions of Siglec-9 on NK cells leading to subsequent tumor cell killing. The disclosures herein support that targeting SSEA-4 on tumor cells can release the immune cell activity by blocking the engagement between SSEA-4 and Siglec-9. Thus SSEA-4 antibody can be used as an immune checkpoint blocker, alone or in combination with other reagents used for oncology.

In one aspect, the present disclosure relates to the use of anti-SSEA-4 antibody in combination with therapeutic oncology agents to treat cancer patients. In certain embodiment, the antibody is OBI-898 (anti-SSEA-4 monoclonal antibody). Exemplary OBI-898 is as described in PCT patent publication (WO2017172990A1), US patent publication (US2018339061A1) patent application, and the contents of each of which are incorporated by reference in its entirety. In one aspect, the present disclosure is based on the discovery that SSEA-4 on cancers can interact with Siglecs on immune cells results in the inactivation of immune cells. Addition of anti-SSEA-4 antibody to block the engagement between SSEA-4 and Siglecs can release the cellular cytotoxicity of immune cells. Therapeutic oncology agents can be combined with anti-SSEA-4 antibody to increase the immune cell cytotoxicity on tumors.

In one aspect, the present invention relates to anti-SSEA-4 antibody combined with therapeutic agents (e.g. therapeutic antibodies and/or chemotherapeutic agents) used for oncology. The disclosure provided examples based on the rationale of administering of anti-SSEA-4 antibody to rescue immune cells inactivation induced by SSEA-4 and Siglecs engagement to improve anticancer efficacy.

Siglec-9 has been described as having immunomodulatory effects on cytokine production. Overexpression of Siglec-9 in a macrophage cell line and concomitant TLR stimulation has been shown to be associated with a decrease in production of proinflammatory cytokines TNF-alpha and IL-6, as well as upregulation of IL-10 (Ando et al. (2008) Biochem. And Biophys. Res. Comm. 369:878-883). It has also been shown that tumor-produced mucins bind to Siglec-9, as well as immature DCs (Ohta et al. (2010) Biochem. and Biophys. Res. Comm. 402: 663-669). In the presence of LPS and mucins, immature DCs produced less IL-12, but IL-10 production was maintained. This suggests that Siglec-9 skews cytokine production from pro-inflammatory to anti-inflammatory, thereby maintaining an immunological state of tolerance as opposed to clearance of offending pathogens, cancer, or other pathologies.

The inhibitory role of Siglec-9 has been further characterized in the function of natural killer cells and regulation of lymphoid cells, such as T cells and neutrophils (Crocker et al. (2012) Ann. N Y Acad. Sci. 1253, 102-111; Pillai et al. (2012) Annu. Rev. Immunol. 30, 357-392; von Gunten and Bochner (2008) Ann. N Y Acad. Sci. 1143, 61-82; Jandus et al. (2014) J. Clin. Invest. 124(4) 1810-1820; Ikehara et al. (2004) J. Biol. Chem. 279:41 43117-43125; and von Gunten et al. (2005) Blood 106(4) 1423-1431). Functional studies in natural killer cells have demonstrated that tumor cells expressing Siglec-9 binding sialic acid ligands inhibit NK cell activation and tumor cell killing. Many human tumors robustly upregulate sialic acid ligands that bind Siglec-9, which enables immune evasion and cancer progression (Jandus et al. (2014) J. Clinic. Invest. 124:4: 1810-1820). It is thought that sialic acid upregulation on tumors facilitates a state of “super self’ that strongly inhibits natural killer cell immunosurveillance (Macauley and Paulson (2014) Nat. Chem. Biol. 10:1: 7-8). In lymphoid lineage cells, Siglec-9 has been shown to negatively regulate T cell receptor signaling via ITIM tyrosine phosphorylation and SHP-1 binding. Downstream TCR signaling molecules ZAP-70 showed reduced phosphorylation on Tyr319 and decreased NFAT transcriptional activity. The inhibitory effects of Siglec-9 on TCR signaling were reduced upon mutation of a conserved Arginine residue in the sialic acid ligand-binding domain (Ikehara et al. (2004) J. Biol. Chem. 279:41 43117-43125). In neutrophils, Siglec-9 engagement mediates cell death via apoptotic and non-apoptotic mechanisms. Neutrophils derived from non-diseased or rheumatoid arthritis and acute septic shock patients underwent Siglec-9 dependent death, demonstrated by antibody crosslinking. Septic or RA-patient-derived neutrophils demonstrated significantly more cell death upon Siglec-9 ligation; this increase could be mimicked by short term pre-incubation with pro-inflammatory cytokines, suggesting that inflammation leads to priming of the Siglec-9 death pathway (Belisle et al. (2010) Mol. Cancer 9:118).

The murine homolog of Siglec-9 is Siglec-E, which is 53% similar. Siglec-E was shown to bind human red blood cells in a sialic acid dependent manner, and functionally like Siglec-9, recruits SHP-1 and SHP-2 via ITIMs to mediate inhibitory signaling in immune cells (Yu et al Biochem. J. (2001) 353, 483-492). In mice, genetic inactivation of Siglec-E does not lead to obvious developmental, histological, or behavioral abnormalities; and Siglec-E-deficient mice breed normally, indicating that Siglec-E is not an essential gene and that its function may be limited to innate immunity (McMillan et al. (2013) Blood 121:11: 2084-2094). Upon challenge of Siglec-E deficient mice with aerosol LPS, increased neutrophil recruitment in the lung was demonstrated, which could be reversed by blockade of the 132-integrin CD11b. The Siglec-E deficient neutrophils were shown to have increased phosphorylation of Syk and p38 MAPK in a CD11b-dependent manner. This suggests that Siglec-E functions to suppress neutrophil recruitment in a model of acute lung inflammation (McMillan et al. (2013) Blood 121:11: 2084-2094). In a syngeneic cancer model, neutrophils from Siglec-E deficient mice enhanced tumor cell killing ex vivo and demonstrated increased ROS production and apoptosis inducing ligands such as TRAIL and FasL (Laubli et al. (2014) PNAS 111 (39) 14211-14216).

In oncology, Siglec-9 has been suggested as a therapeutic target for acute myeloid leukemia as it is expressed on primary AML cells, yet absent from progenitors on numerous patient bone marrow samples (Biedermann et al. (2007) Leuk. Res. 31:2:211-220). In solid cancers, epithelial tumor cells produce heavily glycosylated mucins that bind Siglec-9, suggesting that blocking the increased ligand interactions would be therapeutically beneficial (Ohta et al. (2010) Biochem. and Biophys. Res. Comm. 402: 663-669; Belisle et al. (2010) Mol. Cancer 9:118). Furthermore, robust expression of Siglec-9 ligands and tumor infiltrating Siglec-9+ immune cells were found in histological sections of colorectal, breast, ovarian, non-small lung cell, and prostate cancer (Laubli et al. (2014) PNAS 111 (39) 14211-14216). A naturally occurring Siglec-9 K131Q (A391C) polymorphism (rs16988910) that reduces sialyl ligand binding was found to significantly improve early survival (<2 years) in non-small-cell lung cancer patients, though the effect was lost after 2 years (Laubli et al. (2014) PNAS 111 (39) 14211-14216).

It has recently been proposed that sialyl-glycoproteins expressed on cancer cells transduce ‘activation’ signals into tumor cells via Siglec-9 binding, resulting in degradation of Focal adhesion kinase (FAK) and increased cell motility and invasion (Sabit et al. (2013) J. Biol. Chem. 288(49): 35417-35427). These results suggest that Siglec-9-sialyl ligand interactions not only contribute to inhibitory effects on numerous cell types of the immune system, but could also enhance tumor metastasis via direct effects on cancer cells.

Accordingly, there is a need for therapeutic antibodies that specifically modulate Siglec-9 interactions between Siglec-9 and one or more Siglec-9 ligands, and/or reduce one or more Siglec-9 activities in order to treat one or more diseases, disorders, and conditions associated with undesired Siglec-9 activity.

### Definitions

The practice of the present invention employs, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

As used herein, the term “glycan” refers to a polysaccharide, or oligosaccharide. Glycan is also used herein to refer to the carbohydrate portion of a glycoconjugate, such as a glycoprotein, glycolipid, glycopeptide, glycoproteome, peptidoglycan, lipopolysaccharide, or a proteoglycan. Glycans usually consist solely of O-glycosidic linkages between monosaccharides. For example, cellulose is a glycan (or more specifically a glucan) composed of ß-1,4-linked D-glucose, and chitin is a glycan composed of ß-1,4-linked N-acetyl-D-glucosamine. Glycans can be homopolymers or heteropolymers of monosaccharide residues, and can be linear or branched. Glycans can be found attached to proteins as in glycoproteins and proteoglycans. They are generally found on the exterior surface of cells. O- and N-linked glycans are very common in eukaryotes but may also be found, although less commonly, in prokaryotes. N-Linked glycans are found attached to the R-group nitrogen (N) of asparagine in the sequon. The sequon is a Asn-X-Ser or Asn-X-Thr sequence, where X is any amino acid except praline.

As used herein, the term “antigen” is defined as any substance capable of eliciting an immune response.

As used herein, the term “immunogenicity” refers to the ability of an immunogen, antigen, or vaccine to elicit an immune response.

As used herein, the term “epitope” is defined as the parts of an antigen molecule which contact the antigen binding site of an antibody or a T cell receptor.

As used herein, the term “vaccine” refers to a preparation that contains an antigen, consisting of whole disease-causing organisms (killed or weakened) or components of such organisms, such as proteins, peptides, or polysaccharides, that is used to confer immunity against the disease that the organisms cause. Vaccine preparations can include or exclude any one of natural, synthetic or recombinantly derived preparations. Recombinantly derived preparations can be obtained, for example, by recombinant DNA technology.

As used herein, the term “antigen specific” refers to a property of a cell population such that the supply of a particular antigen, or a fragment of the antigen, results in specific cell proliferation.

As used herein, the term “specific binding,” refers to the interaction between binding pairs (e.g., an antibody and an antigen). In various instances, specific binding can be embodied by an affinity constant of about 10⁻⁶ moles/liter, about 10⁻⁷ moles/liter, or about 10⁻⁸ moles/liter, or less.

The phrase “substantially similar”, “substantially the same”, “equivalent”, or “substantially equivalent”, as used herein, denotes a sufficiently high degree of similarity between two numeric values (for example, one associated with a molecule and the other associated with a reference/comparator molecule) such that one of skill in the art would consider the differences between the two values to be of little or no biological and/or statistical significance within the context of the biological characteristic measured by said values (e.g., Kd values, anti-viral effects, etc.). The differences between said two values is, for example, less than about 50%, less than about 40%, less than about 30%, less than about 20%, and/or less than about 10% as a function of the value for the reference/comparator molecule.

The phrase “substantially reduced,” or “substantially different”, as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values). The differences between said two values are, for example, greater than about 10%, greater than about 20%, greater than about 30%, greater than about 40%, and/or greater than about 50% as a function of the value for the reference/comparator molecule.

“Binding affinity”, as used herein, generally refers to the strength of the sum of total noncovalent interactions between a single binding site of a molecule (e.g., an antibody) and its binding partner (e.g., an antigen). Unless indicated otherwise, as used herein, “binding affinity” refers to the intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described in the following.

In certain embodiments, the “Kd” or “Kd value” according to this invention is measured by a radiolabeled antigen binding assay (RIA) performed with the Fab version of an antibody of interest and its antigen as described by the following assay. Solution binding affinity of Fabs for antigen is measured by equilibrating Fab with a minimal concentration of (125I)-labeled antigen in the presence of a titration series of unlabeled antigen, then capturing bound antigen with an anti-Fab antibody-coated plate (Chen, et al. (1999) J. Mol Biol 293:865-881). To establish conditions for the assay, microtiter plates (Dynex) are coated overnight with 5 μg/mL of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc, Cat #269620), 100 pM or 26 pM [125I]-antigen are mixed with serial dilutions of a Fab of interest (e.g., consistent with assessment of an anti-VEGF antibody, Fab-12, in Presta et al. (1997) Cancer Res. 57:4593-4599). The Fab of interest is then incubated overnight; however, the incubation may continue for a longer period (e.g., 65 hours) to insure that equilibrium is reached. Thereafter, the mixtures are transferred to the capture plate for incubation at room temperature (e.g., for one hour). The solution is then removed and the plate washed eight times with 0.1% Tween-20 in PBS. When the plates have dried, 150 μL/well of scintillant (MicroScint-20; Packard) is added, and the plates are counted on a Topcount gamma counter (Packard) for ten minutes. Concentrations of each Fab that give less than or equal to 20% of maximal binding are chosen for use in competitive binding assays. According to another embodiment the Kd or Kd value is measured by using surface plasmon resonance assays using a BIAcore™-2000 or a BIAcore™-3000 (BIAcore, Inc., Piscataway, N.J.) at 25° C., with immobilized antigen CMS chips at ^{˜}10 response units (RU). Briefly, carboxymethylated dextran biosensor chips (CMS, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (“0.2 μM) before injection at a flow rate of 5 μL/minute to achieve approximately 10 response units (RU) of coupled protein. Following the injection of antigen, 1 M ethanolamine is injected to block unreacted groups. In each experiment, a spot was activated and ethanolamine blocked without immobilizing protein, to be used for reference subtraction. For kinetics measurements, two-fold serial dilutions of Fab (0.78 nM to 500 nM) are injected in PBS with 0.05% Tween 20 (PBST) at 25° C. at a flow rate of approximately 25 μL/min. Association rates (kon) and dissociation rates (koff) are calculated using a simple one-to-one Langmuir binding model (BIAcore Evaluation Software version 3.2) by simultaneously fitting the association and dissociation sensorgrams. The equilibrium dissociation constant (Kd) is calculated as the ratio koff/kon. See, e.g., Chen, Y., et al. (1999) J. Mol Biol 293:865-881. If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

An “on-rate” or “rate of association” or “association rate” or “kon” according to this invention can also be determined with the same surface plasmon resonance technique described above using a BIAcore™-2000 or a BIAcore™-3000 (BIAcore, Inc., Piscataway, N.J.) at 25° C. with immobilized antigen CMS chips at or “association rate” or “kon” according to this invention can also be determined with the same surface plasmon N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/mL (^{˜}0.2 μM) before injection at a flow rate of 5 μL/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% Tween 20 (PBST) at 25° C. at a flow rate of approximately 25 μL/min. Association rates (kon) and dissociation rates (koff) are calculated using a simple one-to-one Langmuir binding model (BIAcore Evaluation Software version 3.2) by simultaneously fitting the association and dissociation sensorgram. The equilibrium dissociation constant (Kd) was calculated as the ratio koff/kon. See, e.g., Chen, Y., et al. (1999) J. Mol Biol 293:865-881. However, if the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-Aminco spectrophotometer (ThermoSpectronic) with a stirred cuvette.

“Antibodies” (Abs) and “immunoglobulins” (Igs), as used herein, are glycoproteins having the same structural characteristics. While antibodies exhibit binding specificity to a specific antigen, immunoglobulins include both antibodies and other antibody-like molecules which generally lack antigen specificity. Polypeptides of the latter kind are, for example, produced at low levels by the lymph system and at increased levels by myelomas.

The terms “antibody” and “immunoglobulin”, as used herein, are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity), and may also include certain antibody fragments, as described in greater detail herein. An antibody can be chimeric, human, humanized, and/or affinity matured.

The “variable region” or “variable domain” of an antibody, as used herein, refers to the amino-terminal domains of heavy or light chain of the antibody. These domains are generally the most variable parts of an antibody and contain the antigen-binding sites.

The term “variable”, as used herein, refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al. (1991) Sequences of Proteins of Immunological Interest, Fifth Edition, National Institute of Health, Bethesda, Md.). The constant domains are not involved directly in binding an antibody to an antigen, but exhibit various effector functions, such as participation of the antibody in antibody-dependent cellular toxicity.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species, one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

The terms “full length antibody,” “intact antibody” and “whole antibody” are used herein interchangeably, to refer to an antibody in its substantially intact form, not antibody fragments as defined below. The terms particularly refer to an antibody with heavy chains that contain the Fc region.

“Antibody fragments”, as used herein, comprise only a portion of an intact antibody, wherein the portion retains at least one, and as many as most or all, of the functions normally associated with that portion when present in an intact antibody. In one embodiment, an antibody fragment comprises an antigen binding site of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half-life modulation, ADCC function and complement binding. In one embodiment, an antibody fragment is a monovalent antibody that has an in vivo half-life substantially similar to an intact antibody. For example, such an antibody fragment may comprise an antigen binding arm linked to an Fc sequence capable of conferring in vivo stability to the fragment.

The term “monoclonal antibody” as used herein refers to an antibody obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. In certain embodiments, the monoclonal antibody may exclude natural sequences. In some aspects, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (e.g., epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al. (1975) Nature, 256: 495; Harlow et al. Antibodies: A Laboratory Manual, (Cold Spring Harbor Laboratory Press, 2nd ed. 1988); Hammerling et al. Monoclonal Antibodies and T-Cell hybridomas 563-681 (Elsevier, N.Y., 1981), recombinant DNA methods (see, e.g., U.S. Pat. No. 4,816,567), phage display technologies (see, e.g., Clackson et al. (1991) Nature, 352: 624-628; Marks et al. (1992) J. Mol. Biol. 222: 581-597; Sidhu et al. (2004) J. Mol. Biol. 338(2): 299-310; Lee et al. (2004) J. Mol. Biol. 340(5): 1073-1093; Fellouse (2004) Proc. Natl. Acad. Sci. USA 101(34): 12467-12472; and Lee et al. (2004) J. Immunol. Methods 284(1-2): 119-132, and technologies for producing human or human-like antibodies in animals that have parts or all of the human immunoglobulin loci or genes encoding human immunoglobulin sequences (see, e.g., WO98/24893; WO96/34096; WO96/33735; WO91/10741; Jakobovits et al., Proc. Natl. Acad. Sci. USA 90: 2551 (1993); Jakobovits et al. (1993) Nature 362: 255-258; Bruggemann et al. (1993) Year in Immunol. 7:33; U.S. Pat. Nos. 5,545,807; 5,545,806; 5,569,825; 5,625,126; 5,633,425; 5,661,016; Marks et al. (1992) Bio. Technology 10: 779-783; Lonberg et al. (1994) Nature 368: 856-859; Morrison (1994) Nature 368: 812-813; Fishwild et al. (1996) Nature Biotechnol. 14: 845-851; Neuberger (1996) Nature Biotechnol. 14: 826 and Lonberg and Huszar (1995) Intern. Rev. Immunol. 13: 65-93.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al. (1984) Proc. Natl. Acad. Sci. USA 81:6851-6855).

Antibodies of the present invention also include chimerized or humanized monoclonal antibodies generated from antibodies of the present invention.

The antibodies can be full-length or can comprise a fragment (or fragments) of the antibody having an antigen-binding portion, including, but not limited to, Fab, F(ab′)₂, Fab′, F(ab)′, Fv, single chain Fv (scFv), bivalent scFv (bi-scFv), trivalent scFv (tri-scFv), Fd, dAb fragment (e.g., Ward et al. (1989) Nature, 341:544-546), an CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention. Bird et al. (1977) Science, 242:423-426. Huston et al. (1988) Proc. Natl. Acad. Sci. USA, 1988, 85:5879-5883.

The antibodies or antigen-binding portions thereof of the present invention may be monospecific, bi-specific or multispecific.

All antibody isotypes are encompassed by the present invention, including IgG (e.g., IgG₁, IgG₂, IgG₃, IgG₄), IgM, (IgA₁, IgA₂), IgD or IgE (all classes and subclasses are encompassed by the present invention). The antibodies or antigen-binding portions thereof may be mammalian (e.g., mouse, human) antibodies or antigen-binding portions thereof. The light chains of the antibody may be of kappa or lambda type.

Thus antibodies of the present invention include in combination with a heavy chain or light chain variable region, a heavy chain or light chain constant region, a framework region, or any portion thereof, of non-murine origin, preferably of human origin, which can be incorporated into an antibody of the present invention.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from a hypervariable region of the recipient are replaced by residues from a hypervariable region of a non-human species (donor antibody) such as mouse, rat, rabbit or nonhuman primate having the desired specificity, affinity, and/or capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al. (1986) Nature 321:522-525; Riechmann et al. (1988) Nature 332:323-329; and Presta (1992) Curr. Op. Struct. Biol. 2:593-596. See also the following review articles and references cited therein: Vaswani and Hamilton (1988) Ann. Allergy, Asthma & Immunol. 1:105-115; Harris (1995) Biochem. Soc. Transactions 23:1035-1038; Hurle and Gross (1994) Curr. Op. Biotech. 5:428-433.

The term “hypervariable region”, “HVR”, or “HV”, when used herein refers to the regions of an antibody variable domain which are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat Complementarity Determining Regions (CDRs) are based on sequence variability and are the most commonly used (Kabat et al. (1991) Sequences of Proteins of Immunological Interest, 5th Ed. Public Health Service, National Institutes of Health, Bethesda, Md.). Chothia refers instead to the location of the structural loops (Chothia and Lesk (1987) J. Mol. Biol. 196:901-917).

“Framework” or “FW” residues, as used herein, are those variable domain residues other than the hypervariable region residues as herein defined.

The term “variable domain residue numbering as in Kabat” or “amino acid position numbering as in Kabat” and variations thereof, refers to the numbering system used for heavy chain variable domains or light chain variable domains of the compilation of antibodies in Kabat et al. Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or HVR of the variable domain. For example, a heavy chain variable domain may include a single amino acid insert (e.g., residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

“Single-chain Fv” or “scFv” antibody fragments, as used herein, comprise the VH and VL domains of antibody, wherein these domains are present in a single polypeptide chain. Generally, the scFv polypeptide further comprises a polypeptide linker between the VH and VL domains which enables the scFv to form the desired structure for antigen binding. For a review of scFv see Pluckthun, in The Pharmacology of Monoclonal Antibodies, vol. 113, Rosenburg and Moore eds., Springer-Verlag, New York, pp. 269-315 (1994).

The term “diabodies”, as used herein, refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO93/1161; and Hollinger et al. (1993) Proc. Natl. Acad. Sci. USA 90: 6444-6448.

A “human antibody”, as used herein, is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured antibody”, as used herein, is one with one or more alterations in one or more HVRs thereof which result in an improvement in the affinity of the antibody for antigen, compared to a parent antibody which does not possess those alteration(s). In one embodiment, an affinity matured antibody has nanomolar or even picomolar affinities for the target antigen. Affinity matured antibodies are produced by procedures known in the art. Marks et al. (1992) Bio/Technology 10:779-783 describes affinity maturation by VH and VL domain shuffling. Random mutagenesis of CDR and/or framework residues is described by: Barbas et al. (1994) Proc Nat. Acad. Sci. USA 91:3809-3813; Schier et al. (1995) Gene 169:147-155; Yelton et al. (1995) J. Immunol. 155:1994-2004; Jackson et al. (1995) J. Immunol. 154(7):3310-9; and Hawkins et al. (1992) J. Mol. Biol. 226:889-896.

A “blocking antibody” or an “antagonist antibody”, as used herein, is one which inhibits or reduces biological activity of the antigen it binds. Certain blocking antibodies or antagonist antibodies substantially or completely inhibit the biological activity of the antigen.

An “agonist antibody”, as used herein, is an antibody which mimics at least one of the functional activities of a polypeptide of interest.

A “disorder”, as used herein, is any condition that would benefit from treatment with an antibody of the invention. This includes chronic and acute disorders or diseases including those pathological conditions which predispose the mammal to the disorder in question. Non-limiting examples of disorders to be treated herein include cancer.

The terms “cell proliferative disorder” and “proliferative disorder”, as used herein, refer to disorders that are associated with some degree of abnormal cell proliferation. In one embodiment, the cell proliferative disorder is cancer.

“Tumor” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous”, as used herein, refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulvar cancer, thyroid cancer, hepatic carcinoma, leukemia and other lymphoproliferative disorders, and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In certain embodiments, antibodies of the invention are used to delay development of a disease or disorder.

As used herein, “antibody-drug conjugates (ADCs)” refers to an antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

As used herein, “T cell surface antigen” refers to an antigen can include representative T cell surface markers known in the art, including T-cell antigen receptor (TcR), which is the principle defining marker of all T-cells which are used by the T-cell for specific recognition of MHC-associated peptide antigens. An exemplar associated with the TcR is a complex of proteins known as CD3, which participate in the transduction of an intracellular signal following TcR binding to its cognate MHC/antigen complex. Other examples of T cell surface antigen can include (or exclude) CD2, CD4, CD5, CD6, CD8, CD28, CD40L and/or CD44.

An “individual” or a “subject”, as used herein, is a vertebrate. In certain embodiments, the vertebrate is a mammal. Mammals include, but are not limited to, farm animals (such as cows), sport animals, pets (such as cats, dogs, and horses), primates, mice and rats. In certain embodiments, the vertebrate is a human.

“Mammal” for purposes of treatment, as used herein, refers to any animal classified as a mammal, including humans, domestic and farm animals, and zoo, sports, or pet animals, such as dogs, horses, cats, cows, etc. In certain embodiments, the mammal is human.

An “effective amount”, as used herein, refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount would be less than the therapeutically effective amount.

The term “cytotoxic agent” as used herein refers to a substance that inhibits or prevents the function of cells and/or causes destruction of cells. The term is intended to include radioactive isotopes (e.g., At211, I131, I125, Y90, Re186, Re188, Sm153, Bi212, P32, Pb212 and radioactive isotopes of Lu), chemotherapeutic agents (e.g., methotrexate, adriamycin, vinca alkaloids, vincristine, vinblastine, etoposide, doxorubicin, melphalan, mitomycin C, chlorambucil, daunorubicin, or other intercalating agents), enzymes, and fragments thereof such as nucleolyticenzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

As used herein, exemplary therapeutic oncology agents include, but are not limited to the chemotherapeutic agents and antibodies. A “chemotherapeutic agent”, as used herein, is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew (1994) Chem. Intl. Ed. Engl., 33: 183-186); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

### Antibodies Targeting SSEA-4

One aspect of the present disclosure features antibody targeting the SSEA-4 related antigens.

Exemplary antibodies include antibody fragments, antibody variants, monoclonal antibodies, polyclonal antibodies, and recombinant antibodies and the like. Antibodies can be generated in mice, rabbits or humans.

The mAb ills is a monoclonal antibody, produced by the hybridoma cell line (ATCC Accession No. PTA-122679). The antibody described herein can contain the same VH and VL chains as antibody ills. Antibodies binding to the same epitope as ills are also within the scope of this disclosure.

Exemplars and their amino acid and nucleic acid structures/sequences are provided below:

TABLE 1
Amino Acid and Nucleotide Sequences of Antibody 1J1s

| SEQ ID | | |
|---|---|---|
| NO | DESCRIPTION | SEQUENCE |
| 1 | 1J15 VH | CAGGTGCAGCTGAAGGAGTCAGGACCTGGCCTGGTGGCGC |
| | nucleotide | CCTCACAGAGCCTGTCCATCACTTGCACTGTCTCTGGGTTT |
| | sequence | TCATTAATCAGCTATGGTGTAGACTGGGTTCGCCAGCCTCC |
| | | AGGAAAGGGTCTGGAGTGGCTGGGAGTAATATGGGGTGGT |
| | | GGAAATACAAATTATAATTCATCTCTCATGTCCAGACTGA |
| | | GCATCAGCAAAGACAACTCCAAGAGCCAAGTTTTCTTAAA |
| | | AATGAACAGTCTGCAAACTGATGACACAGCCATGTACTAC |
| | | TGTGCCAAAACTGGGACCGGATATGCTTTGGAGTACTGGG |
| | | GTCAAGGAACCTCAGTCACCGTCTCCTCC |
| 2 | 1J1s VL | GAAAATGTTCTCACCCAGTCTCCAGCAATCATGTCTGCATC |
| | nucleotide | TCCAGGGGAAAAGGTCACCATGACCTGCAGTGCCAGGTCA |
| | sequence | AGTGTAAGTTACATGCACTGGTACCAGCAGAAGTCAACCG |
| | | CCTCCCCCAAACTCTGGATTTATGACACATCCAAACTGGCT |
| | | TCTGGAGTCCCAGGTCGCTTCAGTGGCAGTGGGTCTGGAA |
| | | ACTCTTACTCTCTCACGATCAGCAGCATGGAGGCTGAAGA |
| | | TGTTGCCACTTATTACTGTTTTCAGGCGAGTGGGTACCCGC |
| | | TCACGTTCGGTGCTGGGACCAAGCTGGAGCTGAAACGG |
| 3 | 1J1s VH amino | QVQLKESGPGLVAPSQSLSITCTVSGFSLISYGVDWVRQPPGK |
| | acid sequence | GLEWLGVIWGGGNTNYNSSLMSRLSISKDNSKSQVFLKMNS |
| | | LQTDDTAMYYCAKTGTGYALEYWGQGTSVTVSS |
| 4 | 1J1s VL amino | ENVLTQSPAIMSASPGEKVTMTCSARSSVSYMHWYQQKSTA |
| | acid sequence | SPKLWIYDTSKLASGVPGRFSGSGSGNSYSLTISSMEAEDVAT |
| | | YYCFQASGYPLTFGAGTKLELKR |
| 5 | 1J1s VL FW1 | ENVLTQSPAIMSASPGEKVTMTC |
| 6 | 1J1s VL CDR1 | SARSSVSYMH |
| 7 | 1J1s VL FW2 | WYQQKSTASPKLWIY |
| 8 | 1J1s VL CDR2 | DTSKLAS |
| 9 | 1J1s VL FW3 | GVPGRFSGSGSGNSYSLTISSMEAEDVATYYC |
| 10 | 1J1s VL CDR3 | FQASGYPLT |
| 11 | 1J1s VL FW4 | FGAGTKLELKR |
| 12 | 1J1s VH FW1 | QVQLKESGPGLVAPSQSLSITCTVS |
| 13 | 1J1s VH CDR1 | GFSLISYGVD |
| 14 | 1J1s VH FW2 | WVRQPPGKGLEWLG |
| 15 | 1J1s VH CDR2 | VIWGGGNTNYNSSLMS |
| 16 | 1J1s VH FW3 | RLSISKDNSKSQVFLKMNSLQTDDTAMYYCAK |
| 17 | 1J1s VH CDR3 | TGTGYALEY |
| 18 | 1J1s VH FW4 | WGQGTSVTVSS |

The mAb 1G1s is a mouse monoclonal antibody, produced by the hybridoma cell line (ATCC Accession No. PTA-122678). The antibodies described herein can contain the same VH and VL chains as antibody 1G1s. Antibodies binding to the same epitope as 1G1s are also within the scope of this disclosure.

Exemplars and their amino acid and nucleic acid structures/sequences are provided below:

TABLE 2
Amino Acid and Nucleotide Sequences of Antibody 1G1s

| SEQ ID | | |
|---|---|---|
| NO | DESCRIPTION | SEQUENCE |
| 19 | 1G1s VH | CAGGTGCAGCTGAAGGAGTCAGGACCTGGCCTGGTGGCGC |
| | nucleotide | CCTCACAGAGCCTGTCCATCACTTGTACTGTCTCTGGGTTT |
| | sequence | TCATTAAGCAGCTATGGTGTAGACTGGGTTCGCCAACCTCC |
| | | AGGAAAGGGTCTGGAGTGGCTGGGAGTAATATGGGGTGGT |
| | | GGAAGCATAAATTATAATTCAGCTCTCATGTCCAGACTGA |
| | | GCATCAGCAAAGACAATTCCAAGAGCCAAATTTTCTTAAA |
| | | AATGAACAGTCTGCAAACTGATGACACAGCCATATACTAC |
| | | TGTACCACACATGAGGATTACGGTCCTTTTGCTTACTGGGG |
| | | CCAAGGGACTCTGGTCACTGTCTCTGCA |
| 20 | 1G1s VL | CAAATTGTTCTCTCCCAGTCTCCAGCAATCCTGTCTGCATC |
| | nucleotide | TCCAGGGGAGAAGGTCACAATGACTTGCAGGGCCAGCTCA |
| | sequence | AGTGTAAGTTACATGCACTGGTACCAGCAGAAGCCAGGAT |
| | | CCTCCCCCAAATCCTGGATTTATGCCACATCCAACCTGGCT |
| | | TCTGGAGTCCCTGCTCGCTTCAGTGGCAGTGGGTCTGGGAC |
| | | CTCTTACTCTCTCACAATCAGCAGAGTGGAGGCTGAAGAT |
| | | GCTGCCACTTATTACTGCCAGCAGTGGGGTAGTTACCCGTG |
| | | GACGTTCGGTGGAGGCACCAAGCTGGAAATCAAACGG |
| 21 | 1G1s VH amino | QVQLKESGPGLVAPSQSLSITCTVSGFSLSSYGVDWVRQPPG |
| | acid sequence | KGLEWLGVIWGGGSINYNSALMSRLSISKDNSKSQIFLKMNS |
| | | LQTDDTAIYYCTTHEDYGPFAYWGQGTLVTVSA |
| 22 | 1G1s VL amino | QIVLSQSPAILSASPGEKVTMTCRASSSVSYMHWYQQKPGSS |
| | acid sequence | PKSWIYATSNLASGVPARFSGSGSGTSYSLTISRVEAEDAATY |
| | | YCQQWGSYPWTFGGGTKLEIKR |
| 23 | 1G1s VL FW1 | QIVLSQSPAILSASPGEKVTMTC |
| 24 | 1G1s VL CDR1 | RASSSVSYMH |
| 25 | 1G1s VL FW2 | WYQQKPGSSPKSWIY |
| 26 | 1G1s VL CDR2 | ATSNLAS |
| 27 | 1G1s VL FW3 | GVPARFSGSGSGTSYSLTISRVEAEDAATYYC |
| 28 | 1G1s VL CDR3 | QQWGSYPWT |
| 29 | 1G1s VL FW4 | FGGGTKLEIKR |
| 30 | 1G1s VH FW1 | QVQLKESGPGLVAPSQSLSITCTVS |
| 31 | 1G1s VH CDR1 | GFSLSSYGVD |
| 32 | 1G1s VH FW2 | WVRQPPGKGLEWLG |
| 33 | 1G1s VH CDR2 | VIWGGGSINYNSALMS |
| 34 | 1G1s VH FW3 | RLSISKDNSKSQIFLKMNSLQTDDTAIYYCTT |
| 35 | 1G1s VH CDR3 | HEDYGPFAY |
| 36 | 1G1s VH FW4 | WGQGTLVTVSA |

The mAb 2F20s is a monoclonal antibody, produced by the hybridoma cell line (ATCC Accession No. PTA-122676). The antibodies described herein can contain the same VH and VL chains as antibody 2F20s. Antibodies binding to the same epitope as 2F20s are also within the scope of this disclosure.

Exemplars and their amino acid and nucleic acid structures/sequences are provided below:

TABLE 3
Amino Acid and Nucleotide Sequences of Antibody 2F20s

| SEQ | | |
|---|---|---|
| ID | | |
| NO | DESCRIPTION | SEQUENCE |
| 37 | 2F20s VH | CAGGTGCAGCTGAAGGAGTCAGGACCTGGCCTGGTGGCG |
| | nucleotide | CCCTCACAGAGCCTGTCCATCACATGCACTGTCTCAGGGT |
| | sequence | TTTCATTAACCAGTTATGGTGTAAGCTGGGCTCGCCAGCC |
| | | TCCAGGAAAGGGTCTGGAGTGGCTGGGAGTAATATGGGG |
| | | TGACGGGAGCACAAATTATCATTCAGCTCTCATATCCAG |
| | | ACTGAGCATCAGCAAGGATAACTCCAAGAGCCAAGTTTT |
| | | CTTAAAACTGAACAGTCTGCAAACTGATGACACAGCCAC |
| | | GTACTACTGTGCCAAACCGGAAAACTGGGACGGCTTCGA |
| | | TGTCTGGGGCCCAGGGACCACGGTCACCGTCTCCTCA |
| 38 | 2F20s VL | CAAATTGTTCTCTCCCAGTCTCCAGCAATCCTGTCTGCAT |
| | nucleotide | CTCCAGGGGAGAAGGTCACAATGACTTGCAGGGCCAGCT |
| | sequence | CAAGTGTAAGTTACATGCACTGGTACCGACAGAAGCCAG |
| | | GATCCTCCCCCAAACCCTGGATTTATGCCACATCCGACCT |
| | | GGCTTCTGGAGTCCCTACTCGCTTCAGTGGCAGTGGGTCT |
| | | GGGACCTCTTACTCTCTCACAATCAGCAGAGTGGAGGCT |
| | | GAAGATGCTGCCACTTATTACTGCCAGCAGTGGAGTAGT |
| | | TACCCGTGGACGTTCGGTGGAGGCACCAAGCTGGAAATC |
| | | AAACGG |
| 39 | 2F20s VH amino | QVQLKESGPGLVAPSQSLSITCTVSGFSLTSYGVSWARQPPG |
| | acid sequence | KGLEWLGVIWGDGSTNYHSALISRLSISKDNSKSQVFLKLNS |
| | | LQTDDTATYYCAKPENWDGFDVWGPGTTVTVSS |
| 40 | 2F20s VL amino | QIVLSQSPAILSASPGEKVTMTCRASSSVSYMHWYRQKPGS |
| | acid sequence | SPKPWIYATSDLASGVPTRFSGSGSGTSYSLTISRVEAEDAA |
| | | TYYCQQWSSYPWTFGGGTKLEIKR |
| 41 | 2F20s VL FW1 | QIVLSQSPAILSASPGEKVTMTC |
| 42 | 2F20s VL CDR1 | RASSSVSYMH |
| 43 | 2F20s VL FW2 | WYRQKPGSSPKPWIY |
| 44 | 2F20s VL CDR2 | ATSDLAS |
| 45 | 2F20s VL FW3 | VPTRFSGSGSGTSYSLTISRVEAEDAATYYC |
| 46 | 2F20s VL CDR3 | QQWSSYPWT |
| 47 | 2F20s VL FW4 | FGGGTKLEIKR |
| 48 | 2F20s VH FW1 | QVQLKESGPGLVAPSQSLSITCTVS |
| 49 | 2F20s VH CDR1 | GFSLTSYGVS |
| 50 | 2F20s VH FW2 | WARQPPGKGLEWLG |
| 51 | 2F20s VH CDR2 | VIWGDGSTNYHSALIS |
| 52 | 2F20s VH FW3 | RLSISKDNSKSQVFLKLNSLQTDDTATYYCAK |
| 53 | 2F20s VH CDR3 | PENWDGFDV |
| 54 | 2F20s VH FW4 | WGPGTTVTVSS |

The humanized anti-SSEA4 (OBI-898) sequences are provided below:

TABLE 4
Anti-SSEA4 (OBI-898) humanized clone sequence

| | |
|---|---|
| Clone name | Amino Acid sequence |

Heavy Chain (V_{H})

| | |
|---|---|
| H4 | QVQLQESGPGLVKPSQTLSLTCTVSGFSLSSYGVDWVRQPPGKGL |
| (SEQ ID No.55) | EWVGVIWGGGNTNYNSSLMSRFTISRDNSKNTLYLQMNSLKTED |
| | TAVYYCAKTGTGYALEYWGQGTTVTVSS |
| H4-16 | QVKLKESGPGLVKPTQTLTLTCTVSGFSLSSYGVDWVRQPPGKG |
| (SEQ ID No.56) | LEWVGVIWGGGNTNYNSSLMSRFTISRDNSKNTLYLQMNSLKTE |
| | DTAVYYCAKTGTGYALEYWGQGTTVTVSS |
| H4-16-N565 | QVKLKESGPGLVKPTQTLTLTCTVSGFSLSSYGVDWVRQPPGKG |
| (SEQ ID No.57) | LEWVGVIWGGGSTNYNSSLMSRFTISRDNSKNTLYLQMNSLKTE |
| | DTAVYYCAKTGTGYALEYWGQGTTVTVSS |
| H4-16-N56Q | QVKLKESGPGLVKPTQTLTLTCTVSGFSLSSYGVDWVRQPPGKG |
| (SEQ ID No.58) | LEWVGVIWGGGQTNYNSSLMSRFTISRDNSKNTLYLQMNSLKTE |
| | DTAVYYCAKTGTGYALEYWGQGTTVTVSS |
| H4-16-N58Y | QVKLKESGPGLVKPTQTLTLTCTVSGFSLSSYGVDWVRQPPGKG |
| (SEQ ID No.59) | LEWVGVIWGGGNTYYNSSLMSRFTISRDNSKNTLYLQMNSLKTE |
| | DTAVYYCAKTGTGYALEYWGQGTTVTVSS |
| H4-16-K3T-N565 | QVTLKESGPGLVKPTQTLTLTCTVSGFSLSSYGVDWVRQPPGKGL |
| (SEQ ID No.60) | EWVGVIWGGGSTNYNSSLMSRFTISRDNSKNTLYLQMNSLKTED |
| | TAVYYCAKTGTGYALEYWGQGTTVTVSS |
| H4-16-K3T-N56Q | QVTLKESGPGLVKPTQTLTLTCTVSGFSLSSYGVDWVRQPPGKGL |
| (SEQ ID No.61) | EWVGVIWGGGQTNYNSSLMSRFTISRDNSKNTLYLQMNSLKTED |
| | TAVYYCAKTGTGYALEYWGQGTTVTVSS |
| H4-16-K3T-N58Y | QVTLKESGPGLVKPTQTLTLTCTVSGFSLSSYGVDWVRQPPGKGL |
| (SEQ ID No.62) | EWVGVIWGGGNTYYNSSLMSRFTISRDNSKNTLYLQMNSLKTED |
| | TAVYYCAKTGTGYALEYWGQGTTVTVSS |
| H4-4 | QVTLKESGPALVKPTQTLTLTCTVSGFSLSSYGVDWVRQPPGKGL |
| (SEQ ID No.63) | EWVGVIWGGGNTNYNSSLMSRFTISRDNSKNTLYLQMNSLKTED |
| | TAVYYCAKTGTGYALEYWGQGTTVTVSS |
| H4-14 | QVKLKESGPALVKPSQTLTLTCTVSGFSLSSYGVDWVRQPPGKGL |
| (SEQ ID No.64) | EWVGVIWGGGNTNYNSSLMSRFTISRDNSKNTLYLQMNSLKTED |
| | TAVYYCAKTGTGYALEYWGQGTTVTVSS |
| H4-18 | QVKLKESGPGLVKPSQTLTLTCTVSGFSLSSYGVDWVRQPPGKGL |
| (SEQ ID No.65) | EWVGVIWGGGNTNYNSSLMSRFTISRDNSKNTLYLQMNSLKTED |
| | TAVYYCAKTGTGYALEYWGQGTTVTVSS |
| H4-19 | QVKLQESGPALVKPSQTLTLTCTVSGFSLSSYGVDWVRQPPGKGL |
| (SEQ ID No.66) | EWVGVIWGGGNTNYNSSLMSRFTISRDNSKNTLYLQMNSLKTED |
| | TAVYYCAKTGTGYALEYWGQGTTVTVSS |
| HCDR1 | GFSLSSYGVDW |
| (SEQ ID No.67) | |
| HCDR2 | VIWGGGNTNYNSSLMSR |
| (SEQ ID No.68) | |
| HCDR3 | TGTGYALE |
| (SEQ ID No.69) | |

Light Chain (V_{L})

| | |
|---|---|
| vK1 | DIQMTQSPSSLSASVGDRVTITCSARSSVSYMHWYQQKPGKVPK |
| (SEQ ID No. 70) | LLIYDTSKLASGVPSRFSGSGSGTDFTLTISSLQPEDVATYYCFQ |
| | ASGYPLTFGGGTKVEIKR |
| Vk2 | EIVLTQSPATLSLSPGERATLSCSARSSVSYMHWYQQKPGQAPRL |
| (SEQ ID No.71) | LIYDTSKLASGIPARFSGSGSGTDFTLTISSLEPEDFAVYYCFQA |
| | SGYPLTFGGGTKVEIKR |
| LCDR1 | SARSSVSYMH |
| (SEQ ID No.72) | |
| LCDR2 | DTSKLAS |
| (SEQ ID No.73) | |
| LCDR3 | FQASGYPLT |
| (SEQ ID No.74) | |

One aspect of the present disclosure features the new antibodies specific to SSEA-4. The anti-SSEA-4 antibody binds to Neu5Acα2→3Galβ1→3GalNAcrβ1→3Galα1→4Galβ1→4Glcβ1 (SSEA-4 hexasaccharide).

Any of the antibodies described herein can be a full length antibody or an antigen-binding fragment thereof. In some examples, the antigen binding fragment is a Fab fragment, a F(ab′)2 fragment, or a single-chain Fv fragment. In some examples, the antigen binding fragment is a Fab fragment, a F(ab′)2 fragment, or a single-chain Fv fragment. In some examples, the antibody is a human antibody, a humanized antibody, a chimeric antibody, or a single-chain antibody.

Any of the antibodies described herein has one or more characteristics of: (a) is a recombinant antibody, a monoclonal antibody, a chimeric antibody, a humanized antibody, a human antibody, an antibody fragment, a bispecific antibody, a monospecific antibody, a monovalent antibody, an IgG₁ antibody, an IgG₂ antibody, or derivative of an antibody; (b) is a human, murine, humanized, or chimeric antibody, antigen-binding fragment, or derivative of an antibody; (c) is a single-chain antibody fragment, a multibody, a Fab fragment, and/or an immunoglobulin of the IgG, IgM, IgA, IgE, IgD isotypes and/or subclasses thereof (d) has one or more of the following characteristics: (i) mediates ADCC and/or CDC of cancer cells; (ii) induces and/or promotes apoptosis of cancer cells; (iii) inhibits proliferation of target cells of cancer cells; (iv) induces and/or promotes phagocytosis of cancer cells; and/or (v) induces and/or promotes the release of cytotoxic agents; (e) specifically binds the tumor-associated carbohydrate antigen, which is a tumor-specific carbohydrate antigen; (f) does not bind an antigen expressed on non-cancer cells, non-tumor cells, benign cancer cells and/or benign tumor cells; and/or (g) specifically binds a tumor-associated carbohydrate antigen expressed on cancer stem cells and on normal cancer cells.

Preferably the binding of the antibodies to their respective antigens is specific. The term “specific” is generally used to refer to the situation in which one member of a binding pair will not show any significant binding to molecules other than its specific binding partner (s) and e.g. has less than about 30%, preferably 20%, 10%, or 1% cross-reactivity with any other molecule other than those specified herein.

The antibodies are suitable bind to the target epitopes with a high affinity (low KD value), and preferably KD is in the nanomolar range or lower. Affinity can be measured by methods known in the art, such as, for example; surface plasmon resonance.

The anti-SSEA-4 antibodies of the invention permit the sensitive and specific detection of the epitopes in straightforward and routine biomolecular assays such as immunoprecipitations, ELISAs, or immunomicroscopy without the need for mass spectrometry or genetic manipulation. In turn, this provides a significant advantage in both observing and elucidating the normal functioning of these pathways and in detecting when the pathways are functioning aberrantly.

In another aspect, the anti-SSEA-4 antibodies of the invention find utility as reagents for detection of cancer states in various cell types and tissues.

In yet another aspect, the present anti-SSEA-4 antibodies are useful for the development of SSEA-4 antagonists with blocking activity patterns similar to those of the subject antibodies of the invention. For example, anti-SSEA-4 antibodies of the invention can be used to determine and identify other antibodies that have the same SSEA-4 binding characteristics and/or capabilities of blocking SSEA-4 pathways.

As a further example, anti-SSEA-4 antibodies of the invention can be used to identify other anti-SSEA-4 antibodies that bind substantially the same antigenic determinant(s) of SSEA-4 as the antibodies exemplified herein, including linear and conformational epitopes.

The anti-SSEA-4 antibodies of the invention can be used in assays based on the physiological pathways in which SSEA-4 is involved to screen for small molecule antagonists of SSEA-4 which will exhibit similar pharmacological effects in blocking the binding of one or more binding partners to SSEA-4 as the antibody does.

### Pharmaceutical Formulations

In one embodiment, the present invention provides pharmaceutical compositions comprising an antibody or antigen-binding portion thereof described herein, and a pharmaceutically acceptable carrier. In another embodiment, the pharmaceutical composition comprises a nucleic acid encoding the present antibody or antigen-binding portion thereof, and a pharmaceutically acceptable carrier. Pharmaceutically acceptable carriers include any and all solvents, dispersion media, isotonic and absorption delaying agents, and the like that are physiologically compatible. In one embodiment, the composition is effective to inhibit cancer cells in a subject.

Routes of administration of the present pharmaceutical compositions include, but are not limited to, intravenous, intramuscular, intranasal, subcutaneous, oral, topical, subcutaneous, intradermal, transdermal, subdermal, parenteral, rectal, spinal, or epidermal administration.

The pharmaceutical compositions of the present invention can be prepared as injectables, either as liquid solutions or suspensions, or as solid forms which are suitable for solution or suspension in liquid vehicles prior to injection. The pharmaceutical composition can also be prepared in solid form, emulsified or the active ingredient encapsulated in liposome vehicles or other particulate carriers used for sustained delivery. For example, the pharmaceutical composition can be in the form of an oil emulsion, water-in-oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of the pharmaceutical composition.

The present antibodies or antigen-binding portions thereof are formulated into pharmaceutical compositions for delivery to a mammalian subject. The pharmaceutical composition is administered alone, and/or mixed with a pharmaceutically acceptable vehicle, excipient or carrier. Suitable vehicles are, for example, water, saline, dextrose, glycerol, ethanol, or the like, and combinations thereof. In addition, the vehicle can contain minor amounts of auxiliary substances such as wetting or emulsifying agents, pH buffering agents, or adjuvants. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds include wetting agents, emulsifying agents, dispersing agents or preservatives. See, for example, the 21^{st} edition of Remington's Pharmaceutical Science, Mack Publishing Company, Easton, Pa. (“Remington's”). The pharmaceutical compositions of the present invention can also include ancillary substances, such as pharmacological agents, cytokines, or other biological response modifiers.

Furthermore, the pharmaceutical compositions can be formulated into pharmaceutical compositions in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Actual methods of preparing such dosage forms are known, or will be apparent, to those skilled in the art. See, for example, Remington's Pharmaceutical Sciences, Mack Publishing Company, Easton, Pa., 21^{st} edition.

Pharmaceutical compositions can be administered in a single dose treatment or in multiple dose treatments on a schedule and over a time period appropriate to the age, weight, and condition of the subject, the particular composition used, and the route of administration, whether the pharmaceutical composition is used for prophylactic or curative purposes, etc. For example, in one embodiment, the pharmaceutical composition according to the invention is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of an antibody according to the invention, i.e., the period of time over which the pharmaceutical composition is administered, can vary, depending on any of a variety of factors, e.g., subject response, etc. For example, the pharmaceutical composition can be administered over a period of time ranging from about one or more seconds to one or more hours, one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

For ease of administration and uniformity of dosage, oral or parenteral pharmaceutical compositions in dosage unit form may be used. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The data obtained from the cell culture assays and animal studies can be used in formulating a range of dosage for use in humans. In one embodiment, the dosage of such compounds lies within a range of circulating concentrations that include the ED₅₀ with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Sonderstrup, Springer, Sem. Immunopathol. 25: 35-45, 2003. Nikula et al. (2000) Inhal. Toxicol. 4(12): 123-53.

An exemplary, non-limiting range for a therapeutically or prophylactically effective amount of an antibody or antigen-binding portion of the invention is from about 0.001 to about 60 mg/kg body weight, about 0.01 to about 30 mg/kg body weight, about 0.01 to about 25 mg/kg body weight, about 0.5 to about 25 mg/kg body weight, about 0.1 to about 20 mg/kg body weight, about 10 to about 20 mg/kg body weight, about 0.75 to about 10 mg/kg body weight, about 1 to about 10 mg/kg body weight, about 2 to about 9 mg/kg body weight, about 1 to about 2 mg/kg body weight, about 3 to about 8 mg/kg body weight, about 4 to about 7 mg/kg body weight, about 5 to about 6 mg/kg body weight, about 8 to about 13 mg/kg body weight, about 8.3 to about 12.5 mg/kg body weight, about 4 to about 6 mg/kg body weight, about 4.2 to about 6.3 mg/kg body weight, about 1.6 to about 2.5 mg/kg body weight, about 2 to about 3 mg/kg body weight, or about 10 mg/kg body weight.

The pharmaceutical composition is formulated to contain an effective amount of the present antibody or antigen-binding portion thereof, wherein the amount depends on the animal to be treated and the condition to be treated. In one embodiment, the present antibody or antigen-binding portion thereof is administered at a dose ranging from about 0.01 mg to about 10 g, from about 0.1 mg to about 9 g, from about 1 mg to about 8 g, from about 2 mg to about 7 g, from about 3 mg to about 6 g, from about 10 mg to about 5 g, from about 20 mg to about 1 g, from about 50 mg to about 800 mg, from about 100 mg to about 500 mg, from about 0.01 μg to about 10 mg, from about 0.05 μg to about 1.5 mg, from about 10 μg to about 1 mg protein, from about 30 μg to about 500 μg, from about 40 μg to about 300 μg, from about 0.1 μg to about 200 μg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy and can be determined by one of ordinary skill in the art without undue experimentation.

Therapeutic formulations comprising an antibody of the invention are prepared for storage by mixing the antibody having the desired degree of purity with optional physiologically acceptable carriers, excipients or stabilizers (Remington's Pharmaceutical Sciences 16^{th} edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (e.g., octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, including, but not limited to those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethyl-cellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### Uses

An antibody of the invention may be used in, for example, in vitro, ex vivo, and in vivo therapeutic methods. Antibodies of the invention can be used as an antagonist to partially or fully block the specific antigen activity in vitro, ex vivo and/or in vivo. Moreover, at least some of the antibodies of the invention can neutralize antigen activity from other species. Accordingly, antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus and rhesus, pig or mouse). In one embodiment, an antibody of the invention can be used for inhibiting antigen activities by contacting the antibody with the antigen such that antigen activity is inhibited. In one embodiment, the antigen is a human protein molecule.

In one embodiment, an antibody of the invention can be used in a method for inhibiting an antigen in a subject suffering from a disorder in which the antigen activity is detrimental, comprising administering to the subject an antibody of the invention such that the antigen activity in the subject is inhibited. In one embodiment, the antigen is a human protein molecule and the subject is a human subject. Alternatively, the subject can be a mammal expressing the antigen with which an antibody of the invention binds. Still further the subject can be a mammal into which the antigen has been introduced (e.g., by administration of the antigen or by expression of an antigen transgene). An antibody of the invention can be administered to a human subject for therapeutic purposes. Moreover, an antibody of the invention can be administered to a non-human mammal expressing an antigen with which the antibody cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration). Antibodies of the invention can be used to treat, inhibit, delay progression of, prevent/delay recurrence of, ameliorate, or prevent diseases, disorders or conditions associated with abnormal expression and/or activity of SSEA-4s and SSEA-4ated proteins, including but not limited to cancer, muscular disorders, ubiquitin-pathway-related genetic disorders, immune/inflammatory disorders, neurological disorders, and other ubiquitin pathway-related disorders.

Antibodies of the invention can be used either alone or in combination with other compositions in a therapy. For instance, an antibody of the invention may be co-administered with another antibody, and/or adjuvant/therapeutic agents (e.g., steroids). For instance, an antibody of the invention may be combined with an anti-inflammatory and/or antiseptic in a treatment scheme, e.g. in treating any of the diseases described herein, including cancer, muscular disorders, ubiquitin-pathway-related genetic disorders, immune/inflammatory disorders, neurological disorders, and other ubiquitin pathway-related disorders. Such combined therapies noted above include combined administration (where the two or more agents are included in the same or separate formulations), and separate administration, in which case, administration of the antibody of the invention can occur prior to, and/or following, administration of the adjunct therapy or therapies.

An antibody of the invention (and adjunct therapeutic agent) can be administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Dosing can be by any suitable route, for example, by injections (e.g., intravenous or subcutaneous injections), depending in part on whether the administration is brief or chronic.

The location of the binding target of an antibody of the invention may be taken into consideration in preparation and administration of the antibody. When the binding target is an intracellular molecule, certain embodiments of the invention provide for the antibody or antigen-binding fragment thereof to be introduced into the cell where the binding target is located. In one embodiment, an antibody of the invention can be expressed intracellularly as an intrabody. The term “intrabody,” as used herein, refers to an antibody or antigen-binding portion thereof that is expressed intracellularly and that is capable of selectively binding to a target molecule, as described in Marasco, Gene Therapy 4: 11-15 (1997); Kontermann, Methods 34: 163-170 (2004); U.S. Pat. Nos. 6,004,940 and 6,329,173; U.S. Patent Application Publication No. 2003/0104402, and PCT Publication No. WO2003/077945. Intracellular expression of an intrabody is effected by introducing a nucleic acid encoding the desired antibody or antigen-binding portion thereof (lacking the wild-type leader sequence and secretory signals normally associated with the gene encoding the antibody or antigen-binding fragment) into a target cell. Any standard method of introducing nucleic acids into a cell may be used, including, but not limited to, microinjection, ballistic injection, electroporation, calcium phosphate precipitation, liposomes, and transfection with retroviral, adenoviral, adeno-associated viral and vaccinia vectors carrying the nucleic acid of interest. One or more nucleic acids encoding all or a portion of an anti-SSEA-4 antibody of the invention can be delivered to a target cell, such that one or more intrabodies are expressed which are capable of intracellular binding to a SSEA-4 and modulation of one or more SSEA-4-mediated cellular pathways.

The antibody composition of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody can be an initial candidate dosage for administration to the patient, whether, for example, by one or more separate administrations, or by continuous infusion. One typical daily dosage might range from about 1 μg for the prevention or treatment of disease, the appropriate dosage of an antibody of the invention (with several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

### Therapeutic Applications

Described herein are therapeutic methods that include administering to a subject in need of such treatment a therapeutically effective amount of a composition that includes one or more antibodies described herein.

In certain embodiments, the subject (e.g., a human patient) in need of the treatment is diagnosed with, suspected of having, or at risk for cancer. Examples of the cancer include, but are not limited to, sarcoma, skin cancer, leukemia, lymphoma, brain cancer, lung cancer, breast cancer, oral cancer, esophagus cancer, stomach cancer, liver cancer, bile duct cancer, pancreas cancer, colon cancer, kidney cancer, cervix cancer, ovary cancer and prostate cancer. In certain embodiments, the cancer is sarcoma, skin cancer, leukemia, lymphoma, brain cancer, lung cancer, breast cancer, ovarian cancer, prostate cancer, colon cancer, or pancreas cancer. In some preferred embodiments, the cancer is brain cancer or glioblastoma multiforme (GBM) cancer. In some aspects the subject has a tumor expressing an SSEA-4 antigen.

In some embodiments, the antibody is capable of targeting SSEA-4-expressing cancer cells by binding to SSEA-4 on the cancer or tumor cells. In certain embodiments, the antibody is capable of disrupting or inhibiting binding of SSEA-4 on cancer/tumor cells to an immunomasking checkpoint inhibitor, which may, in some embodiments, be Siglec-9 expressed on a cytotoxic or cytostatic immune cell. In certain embodiments, disrupting or inhibiting binding of SSEA-4 on cancer/tumor cells to an immunomasking checkpoint inhibitor on a cytotoxic immune cell activates an innate cytotoxic response which kills the cancer/tumor cell and/or inhibits the growth or division of the cancer/tumor cell. In some embodiments the immunomasking checkpoint inhibitor excludes Siglec 7.

The treatment results in reduction of tumor size, elimination of malignant cells, prevention of metastasis, prevention of relapse, reduction or killing of disseminated cancer, prolongation of survival and/or prolongation of time to tumor cancer progression.

In certain embodiments, the treatment further comprises administering an additional therapy to said subject prior to, during or subsequent to said administering of the antibodies. In certain embodiments, the additional therapy is treatment with a chemotherapeutic agent. In certain embodiments, the additional therapy is radiation therapy.

The methods of the invention are particularly advantageous in treating and preventing early stage tumors, thereby preventing progression to the more advanced stages resulting in a reduction in the morbidity and mortality associated with advanced cancer. The methods of the invention are also advantageous in preventing the recurrence of a tumor or the regrowth of a tumor, for example, a dormant tumor that persists after removal of the primary tumor, or in reducing or preventing the occurrence of a tumor.

In certain embodiments, the methods as disclosed herein are useful for the treatment or prevention of a cancer, for example where a cancer is characterized by increased Globo H, SSEA-3 and/or SSEA-4 expression. In certain embodiments the cancer comprises a cancer stem cell. In certain embodiments, the cancer is a pre-cancer, and/or a malignant cancer and/or a therapy resistant cancer. In certain embodiments, the cancer is a brain cancer.

For the methods of the invention, the cancer may be a liquid tumor, e.g., such as leukemia and lymphoma, solid tumor, for example, breast cancer, colorectal cancer, rectal cancer, lung cancer, renal cell cancer, a glioma (e.g., anaplastic astrocytoma, anaplastic oligoastrocytoma, anaplastic oligodendroglioma, glioblastoma multiforme (GBM)), kidney cancer, prostate cancer, liver cancer, pancreatic cancer, soft-tissue sarcoma, carcinoid carcinoma, head and neck cancer, melanoma, and ovarian cancer. In one embodiment, the cancer is a brain cancer or GBM. To practice the method disclosed herein, an effective amount of the pharmaceutical composition/formulation described above, containing at least one antibody described herein, can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having cancer, which include, but not limited to, breast cancer, lung cancer, esophageal cancer, rectal cancer, biliary cancer, liver cancer, buccal cancer, gastric cancer, colon cancer, nasopharyngeal cancer, kidney cancer, prostate cancer, ovarian cancer, cervical cancer, endometrial cancer, pancreatic cancer, testicular cancer, bladder cancer, head and neck cancer, oral cancer, neuroendocrine cancer, adrenal cancer, thyroid cancer, bone cancer, skin cancer, basal cell carcinoma, squamous cell carcinoma, melanoma, or brain tumor. A subject having cancer can be identified by routine medical examination.

“An effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy, if any, the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of cancer. Alternatively, sustained continuous release formulations of the antibodies described herein may be appropriate. Various formulations and devices for achieving sustained release are known in the art.

In one example, dosages for an antibody as described herein may be determined empirically in individuals who have been given one or more administration(s) of the antibody. Individuals are given incremental dosages of the antibody. To assess efficacy of the antibody, an indicator of the disease (e.g., cancer) can be followed according to routine practice.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate cancer, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In certain embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In certain embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen, including the antibody used can vary over time.

For the purpose of the present disclosure, the appropriate dosage of an antibody described herein will depend on the specific antibody (or compositions thereof) employed, the type and severity of the cancer, whether the antibody is administered for preventive or therapeutic purposes, previous therapy, the patient's clinical history and response to the antibody, and the discretion of the attending physician. The administration of the antibodies described herein may be essentially continuous over a preselected period of time or may be in a series of spaced dose, e.g., either before, during, or after developing cancer.

As used herein, the term “treating” refers to the application or administration of a composition including one or more active agents to a subject, who has cancer, a symptom of cancer, or a predisposition toward cancer, with the purpose to cure, heal, alleviate, relieve, alter, remedy, ameliorate, improve, or affect cancer, the symptom of cancer, or the predisposition toward cancer.

Alleviating cancer includes delaying the development or progression of cancer, or reducing cancer severity. Alleviating cancer does not necessarily require curative results. As used therein, “delaying” the development of cancer means to defer, hinder, slow, retard, stabilize, and/or postpone progression of cancer. This delay can be of varying lengths of time, depending on the history of cancer and/or individuals being treated. A method that “delays” or alleviates the development of cancer, or delays the onset of cancer, is a method that reduces probability (the risk) of developing one or more symptoms of cancer in a given time frame and/or reduces extent of the symptoms in a given time frame, when compared to not using the method. Such comparisons are typically based on clinical studies, using a number of subjects sufficient to give a statistically significant result.

“Development” or “progression” of cancer means initial manifestations and/or ensuing progression of cancer. Development of cancer can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of cancer includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

### EXAMPLES

### Example 1. ELISA Binding Assay of Siglec-9 and SSEA-4 Ceramide

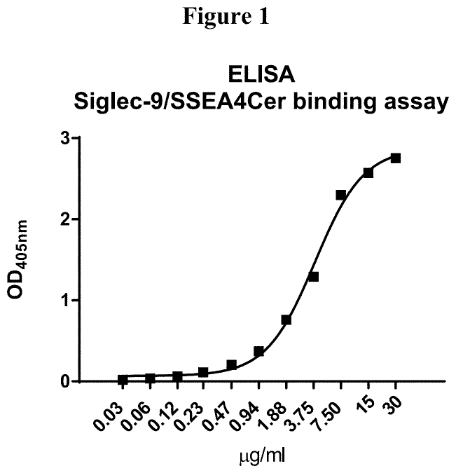

SSEA-4 ceramide (0.2 μg) dissolved in ethanol was coated to each well of a 96-well plate on ice, and the coated plate was incubated at room temperature overnight. Plate was blocked with 100 μL of blocking buffer per well and incubated at room temperature (25° C.) for 1 hour. Following the blocking step, blocking buffer was removed by aspiration and wash each well 3 times with 200 μL of wash buffer (1×TBS with 0.05% Tween20). Transfer 50 μL of Siglec-9 2× serial diluted from 30 μg/mL by sample diluent (1% BSA in 1×TBS with 0.05% tween 20) to the indicated wells of the plate, then incubating the plate at room temperature for 2 hours. After the incubation, the unbounded Siglec-9 was removed by aspiration and the wells were washed 3 times with 200 μL of wash buffer. 50 μL of secondary antibody (anti-human IgG Fc-AP) 1:200 diluted by sample diluent was added to each well of the plate and incubated at room temperature for 1 hour. Secondary antibody was aspirated after the incubation was completed; all wells were washed four times with 200 μL of wash buffer. 100 μL substrate solution was added into each well and developed for 20 minutes at 37° C. The reaction was stopped by the addition of 50 μL of stop solution. The plate was briefly mixed and read at 405 nm by the ELISA plate reader. FIG. 1 showed that human Siglec-9 can bind to SSEA-4 ceramide in ELISA binding assay.

### Example 2. Increase the Siglec-9 Binding on Tumor Cells by Exogeneous of SSEA-4 Ceramide

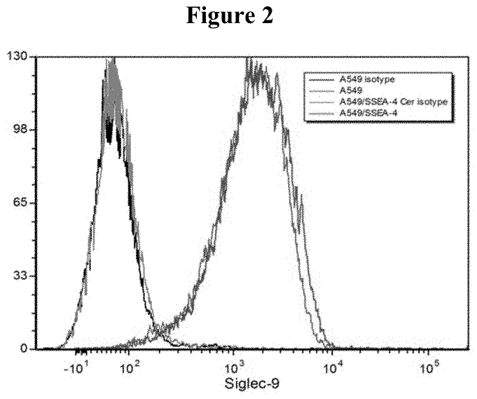

Human A549 lung cancer cells were pre-incubated with 20 μM SSEA-4 ceramide (SSEA4Cer) for 18-24 hours in 24-well culture plate. Cells were collected and centrifuged then stained with Siglec-9 at 4° C. for 30 minutes. Samples were washed and centrifuged then stained with anti-human IgG labeled with FITC at 4° C. for 30 minutes. Cells were then washed and collected. The binding of Siglec-9 on A549 was analyzed by FACS CANTO II. FIG. 2 showed that exogeneous of SSEA4Cer slightly increase the Siglec-9 binding to A549 lung cancer cell.

### Example 3. Reduce the Siglec-9 Binding on Tumor Cells by Adding of Anti-SSEA-4 Fab

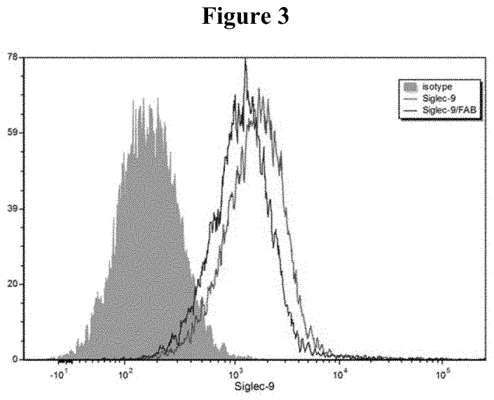

Human MDA-MB-231 breast cancer cell was incubated with OBI-898 Fab, an anti-SSEA-4 antibody, in staining buffer at 4° C. for 30 minutes. Samples were washed and centrifuged then stained with Siglec-9 at 4° C. for 30 minutes. Samples were washed and centrifuged then stained with anti-human IgG labeled with FITC at 4° C. for 30 minutes. Cells were then washed and collected. The binding of Siglec-9 on MDA-MB-231 was analyzed by FACS CANTO II. FIG. 3 showed that OBI-898 Fab can reduce the Siglec-9 binding to MDA-MB-231 breast cancer cell.

### Example 4. Binding of SSEA-4 by Anti-SSEA-4 Fab on Breast Cancer Cell Enhanced the Cellular Cytotoxicity of Human PBMCs

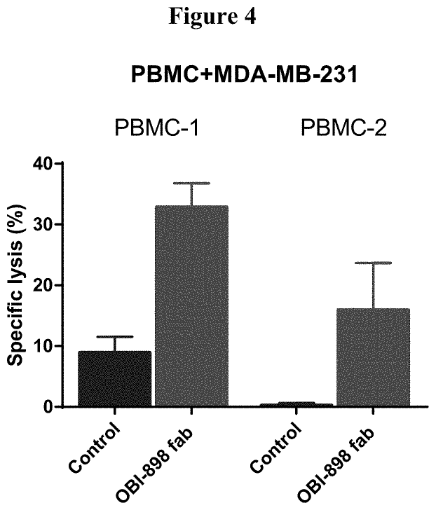

Target human breast cancer cell line, MDA-MB-231, was labeled with DELFIA (Cat # PK-AD0116) in accordance with the technical manual provided by PerkinElmer. After labeling, 5×10³ cells were incubated with or without 20 μg/mL OBI-898 Fab at 37° C. for 1 hour. Then 5×10⁵ human PBMC was added and co-incubated at 37° C. for 2 hours. The assay plate was centrifuged then transfer 20 μL of the supernatant to a flat bottom 96 well plate. 200 μL of Eu-solution was added and incubate for 15 minutes at room temperature on a shaker. Fluorescence was measured in a time-resolved fluorometer. Specific releasing percentage was calculated by the formulation of [(experimental release-spontaneous release)/(maximum release−spontaneous release)]×100. FIG. 4 showed that blocking of SSEA-4 on tumor by anti-SSEA-4 Fab effectively rescue the cellular cytotoxicity of immune cells to kill breast cancers.

### Example 5. Binding of SSEA-4 by Anti-SSEA-4 Fab on Ovary Cancer Cell Line Enhanced the Cellular Cytotoxicity of Human PBMCs

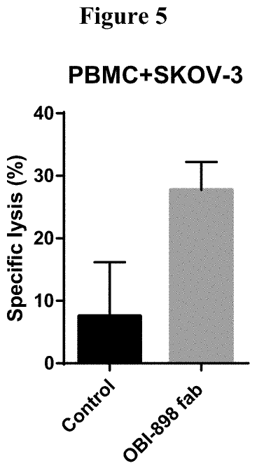

Target human ovary cancer cell line, SKOV-3, was labeled with DELFIA (Cat # PK-AD0116) in accordance with the technical manual provided by PerkinElmer. After labeling, 5×10³ cells were incubated with or without 20 μg/mL anti-SSEA-4 (OBI-898) Fab at 37° C. for 1 hour. Then 5×10⁵ human PBMC was added and co-incubated at 37° C. for 4 hours. The assay plate was centrifuged then transfer 20 μL of the supernatant to a flat bottom 96 well plate. 200 μL of Eu-solution was added and incubate for 15 minutes at room temperature on a shaker. Fluorescence was measured in a time-resolved fluorometer. Specific releasing percentage was calculated by the formulation of [(experimental release-spontaneous release)/(maximum release−spontaneous release)]×100. FIG. 5 showed that blocking of SSEA-4 on tumor by anti-SSEA-4 Fab effectively rescue the cellular cytotoxicity of immune cells to kill ovary cancers.

### Example 6. Binding of SSEA-4 by Anti-SSEA-4 Fab on Ovary Cancer Cell Line Enhanced the Cellular Cytotoxicity of Tecentriq

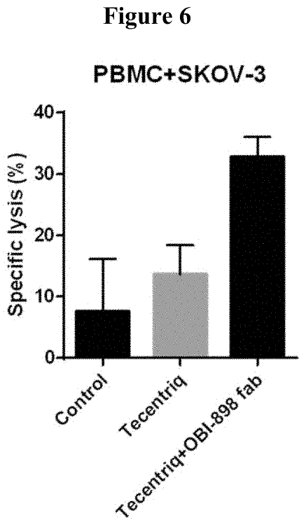

Target human ovary cancer cell line, SKOV-3, was labeled with DELFIA (Cat # PK-AD0116) in accordance with the technical manual provided by PerkinElmer. After labeling, 5×10³ cells were incubated with or without 20 μg/mL anti-SSEA-4 OBI-898 Fab at 37° C. for 1 hour. Then 5×10⁵ human PBMC was added combined with Tecentriq at 20 μg/mL and co-incubated at 37° C. for 4 hours. The assay plate was centrifuged then transfer 20 μL of the supernatant to a flat bottom 96 well plate. 200 μL of Eu-solution was added and incubate for 15 minutes at room temperature on a shaker. Fluorescence was measured in a time-resolved fluorometer. Specific releasing percentage was calculated by the formulation of [(experimental release-spontaneous release)/(maximum release−spontaneous release)]×100. FIG. 6 showed that blocking of SSEA-4 on tumor by anti-SSEA-4 Fab enhance the cytotoxicity of Tecentriq to kill cancer.

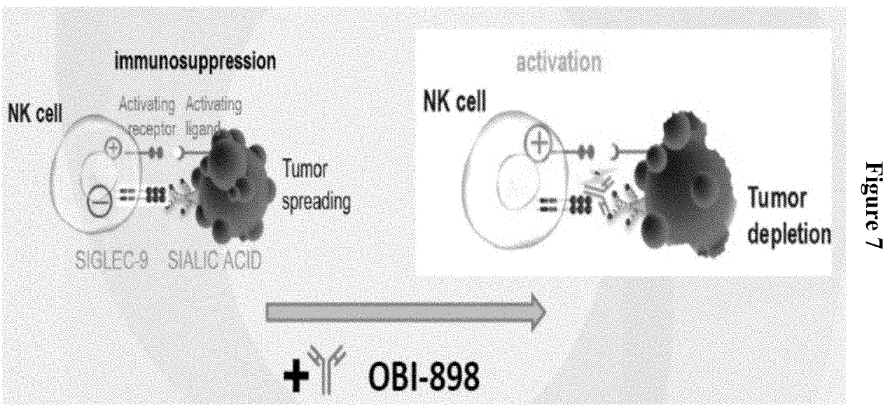

The results in FIG. 7 showed that OBI-898 (anti-SSEA-4 antibody) can block the engagement of Siglec-9 to tumor and release the cytotoxicity of immune cells.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

What is claimed is:

1. A method of treating a subject having a cancer cell expressing a SSEA-4 antigen, the method comprising administering to the subject an effective amount of a pharmaceutical composition comprising an anti-SSEA-4 antibody or a fragment thereof.

2. The method of claim 1, wherein the binding of anti-SSEA-4 antibody to the cancer cell decreases binding interaction between SSEA-4 and Siglec-9.

3. The method of claim 2, wherein the decrease in the binding interaction between SSEA-4 and Siglec-9 results in the decrease binding of Siglec-9 to cancer cells.

4. The method of claim 3, wherein the decrease binding of Siglec-9 to cancer cells induces a release of the immunosuppression (immune-masking) maintained by Siglec-9/SSEA-4 engagement.

5. The method of claim 1, wherein the administering of the anti-SSEA-4 antibody increases the activity of cytotoxic immune cells.

6. The method of claim 5, wherein the cytotoxic immune cell is an monocyte, neutrophil, NK cell, B cell or CD8+ T cell.

7. The method of claim 5, wherein the anti-SSEA-4 antibody is OBI-898.

8. The method of claim 1, wherein the cancer is selected from sarcoma, skin cancer, leukemia, lymphoma, brain cancer, lung cancer, breast cancer, oral cancer, esophagus cancer, stomach cancer, liver cancer, bile duct cancer, pancreas cancer, colon cancer, kidney cancer, cervix cancer, ovary cancer and prostate cancer. In certain embodiments, the cancer is sarcoma, skin cancer, leukemia, lymphoma, brain cancer, lung cancer, breast cancer, ovarian cancer, prostate cancer, colon cancer, or pancreas cancer. In some preferred embodiments, the cancer is brain cancer or glioblastoma multiforme (GBM) cancer.

9. A method of activating an innate cytotoxicity immune response by inhibiting binding of a Siglec-9 expressing cytotoxic immune cell bound to a SSEA-4 antigen on a cancer cell, the method comprising contacting the cytotoxic immune cell-cancer cell complex with an antagonist of SSEA-4; and disrupting or inhibiting binding of Siglec-9 to SSEA-4.

10. The method of claim 9, wherein the antagonist is an SSEA-4 antibody.

11. The method of claim 10, wherein the anti-SSEA-4 antibody is OBI-898.

12. The method of claim 9, wherein the cytotoxic immune cell is an monocyte, neutrophil, NK cell, B cell or CD8+ T cell.

13. The method of claim 9, wherein the cancer is selected from sarcoma, skin cancer, leukemia, lymphoma, brain cancer, lung cancer, breast cancer, oral cancer, esophagus cancer, stomach cancer, liver cancer, bile duct cancer, pancreas cancer, colon cancer, kidney cancer, cervix cancer, ovary cancer and prostate cancer. In certain embodiments, the cancer is sarcoma, skin cancer, leukemia, lymphoma, brain cancer, lung cancer, breast cancer, ovarian cancer, prostate cancer, colon cancer, or pancreas cancer. In some preferred embodiments, the cancer is brain cancer or glioblastoma multiforme (GBM) cancer.

14. A method of treating a subject having a cancer cell expressing a SSEA-4 antigen, the method comprising administering to the subject an effective amount of a pharmaceutical composition comprising an anti-SSEA-4 antibody or a fragment thereof, whereby the binding of Siglec-9 to cancer cells is inhibited.

15. A method of reducing the binding of Siglec-9 to cancer cells, the method comprising administering to the subject an effective amount of a pharmaceutical composition comprising an anti-SSEA-4 antibody or a fragment thereof.

16. A method of treating a subject having a cancer cell expressing a SSEA-4 antigen, the method comprising administering to the subject an effective amount of a pharmaceutical composition comprising an anti-SSEA-4 antibody or a fragment thereof, whereby the activity of the cytotoxic immune cells is activated.

17. A method of increasing the activity of the cytotoxic immune cells in a subject having a cancer, the method comprising administering to the subject an effective amount of a pharmaceutical composition comprising an anti-SSEA-4 antibody or a fragment thereof.

18. The method of claims 14-17, wherein the anti-SSEA-4 antibody is OBI-898.

19. The method of claims 16-17, wherein the cytotoxic cell is an monocyte, neutrophil, NK cell, B cell or CD8+ T cell.

20. The method of claims 14-17, wherein the cancer is selected from sarcoma, skin cancer, leukemia, lymphoma, brain cancer, lung cancer, breast cancer, oral cancer, esophagus cancer, stomach cancer, liver cancer, bile duct cancer, pancreas cancer, colon cancer, kidney cancer, cervix cancer, ovary cancer and prostate cancer. In certain embodiments, the cancer is sarcoma, skin cancer, leukemia, lymphoma, brain cancer, lung cancer, breast cancer, ovarian cancer, prostate cancer, colon cancer, or pancreas cancer. In some preferred embodiments, the cancer is brain cancer or glioblastoma multiforme (GBM) cancer.
